(19) United States
(12) Patent Application Publication
(10) Pub. No.: US 2026/0290611 A1
(43) Pub. Date: Sep. 24, 2026

(54) SYSTEM

(71) Applicant: SoftBank Group Corp., Tokyo (JP)

(72) Inventor: Hirotaka OGAWA, Tokyo (JP)

(73) Assignee: SoftBank Group Corp., Tokyo (JP)

(21) Appl. No.: 19/567,347

(22) Filed: Mar. 16, 2026

(30) Foreign Application Priority Data
Mar. 19, 2025 (JP) 2025-044979

(51) Int. Cl. G16H 50/30; G10L 15/18; G10L 15/26; G10L 25/66; G16H 10/60; G16H 15/00; G16H 40/67

(52) U.S. Cl. CPC G16H 50/30; G10L 15/18; G10L 15/26; G10L 25/66; G16H 10/60; G16H 15/00; G16H 40/67

(57) ABSTRACT

A system includes a processor that is configured to use a prompt to instruct a generative artificial intelligence model to generate one or more questions for checking a health condition of an elderly user, present the generated one or more questions to the elderly user by voice or text and collect one or more answers from the elderly user, and record the collected one or more answers in a database and analyze the recorded answers using data processing techniques.

### CROSS-REFERENCE TO RELATED APPLICATION

This application is based on and claims priority under 35 USC 119 from Japanese Patent Application No. 2025-044979 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional health monitoring for elderly users who live alone or spend substantial time without direct supervision often depends on periodic visits, telephone calls, or manual self-reporting. Such approaches are sporadic, labor-intensive, and highly dependent on the subjective judgment and communication initiative of the elderly user or caregivers. As a result, subtle but important changes in the health condition of the elderly user may not be detected in a timely manner, and opportunities for early intervention may be lost. Furthermore, existing systems that collect health-related information frequently rely on fixed, predetermined question sets that cannot flexibly adapt to the specific context, vocabulary, or behavioral patterns of individual elderly users. This lack of adaptability can reduce user engagement and may lead to incomplete or inaccurate reporting of symptoms. In addition, when abnormal conditions are detected, existing systems may not automatically and promptly notify family members or other relevant parties, thereby delaying support or emergency response. Therefore, there is a need for a system that can automatically generate context-appropriate health-check questions for elderly users, continuously collect and store their answers, analyze the collected answers using data processing techniques, and, when necessary, automatically notify family members based on the analysis results.

### SUMMARY

In order to solve the above problem, a system is provided that comprises a processor configured to use a prompt to instruct a generative artificial intelligence model to generate one or more questions for checking a health condition of an elderly user, present the generated one or more questions to the elderly user by voice or text and collect one or more answers from the elderly user, and record the collected one or more answers in a database and analyze the recorded answers using data processing techniques. By using the prompt to control the generative artificial intelligence model, the processor can dynamically generate questions that are adapted to the individual elderly user, their previous answers, and the current monitoring context, thereby improving the completeness and relevance of the health information obtained. The processor may further be configured to cause the generative artificial intelligence model to generate the one or more questions based on the prompt so that the content, tone, and number of questions can be flexibly adjusted without redesigning the system. In addition, the processor may be configured to automatically transmit a notification to a family member of the elderly user by using a communication technology based on an analysis result obtained by the analysis. Accordingly, when the analysis of the recorded answers indicates a potential abnormality or deterioration in the health condition of the elderly user, the system can automatically and promptly inform the family member, thereby facilitating timely confirmation of the user's safety and early medical or caregiving intervention.

The term “system” refers to an arrangement of one or more hardware and software components that cooperate to perform the functions described in the claims, and may include one or more servers, terminals, storage devices, networks, and communication interfaces.

The term “processor” refers to any hardware component or combination of hardware components capable of executing instructions, including but not limited to a central processing unit (CPU), a graphics processing unit (GPU), a digital signal processor (DSP), a microcontroller, or a programmable logic device, and may represent a single processor or a plurality of processors operating together.

The term “prompt” refers to information, data, or instructions, expressed for example as text or structured data, that is provided to a generative artificial intelligence model in order to cause the model to generate one or more outputs in accordance with the content of the prompt.

The term “generative artificial intelligence model” refers to a machine learning model, such as a neural network, that is trained to generate new data, including natural language text, based on input data such as prompts, and may include large language models, transformer-based models, or other generative models.

The term “question” refers to any interrogative expression or sentence, including follow-up prompts or clarifying inquiries, that is generated for the purpose of eliciting information about a health condition of an elderly user.

The term “one or more questions” refers to a single question or a plurality of questions, which may be generated at one time or sequentially over a period of time.

The term “elderly user” refers to a human user who is of advanced age, for example a senior citizen, and who is the subject of health monitoring by the system.

The term “health condition” refers to a physical, mental, or emotional state of the elderly user, including but not limited to symptoms, discomfort, pain, mood, fatigue, sleep quality, and other parameters relevant to health monitoring.

The term “present” refers to causing information, such as a question, to be output or displayed to the elderly user by a device, including by reproducing audio, showing text or graphics on a screen, or using any other human-perceivable output modality.

The term “voice” refers to an audio output or input modality in which information, such as a question or an answer, is expressed and processed as audible sound.

The term “text” refers to a character-based representation of information, including letters, numbers, symbols, and punctuation, which may be displayed on a screen or otherwise provided to the elderly user in written form.

The term “answer” refers to any response provided by the elderly user to a question, including natural language speech, textual input, or selection input, that contains information relating to the health condition of the elderly user.

The term “one or more answers” refers to a single answer or a plurality of answers, which may be provided in response to one or more questions.

The term “database” refers to any logical or physical data storage system configured to store, manage, and retrieve data, including relational databases, non-relational databases, file-based storage, cloud storage services, or any combination thereof.

The term “record” refers to the act of storing data, such as answers or analysis results, in a database or other storage medium in a manner that allows subsequent retrieval and use.

The term “analyze” refers to processing collected data by applying one or more data processing techniques, such as statistical analysis, pattern recognition, rule-based processing, or machine learning, in order to derive information, metrics, or conclusions.

The term “data processing techniques” refers to algorithms, procedures, or methods used by the processor to transform, evaluate, or interpret data, including but not limited to filtering, aggregating, classifying, scoring, and detecting anomalies.

The term “analysis result” refers to information derived from analyzing recorded answers using data processing techniques, including but not limited to risk scores, detected abnormalities, trends, classifications, and other indicators of the health condition.

The term “communication technology” refers to any hardware, software, or communication protocol used to transmit information between the system and external devices or parties, including but not limited to wired or wireless networks, the Internet, mobile communication networks, email systems, and messaging services.

The term “notification” refers to a message or alert that is generated by the system and transmitted to a recipient, such as a family member, to inform the recipient of a condition, event, or analysis result related to the elderly user.

The term “family member” refers to a person having a familial or caregiver relationship with the elderly user, including but not limited to a relative, guardian, or designated contact person, who is specified in the system as a recipient of notifications.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

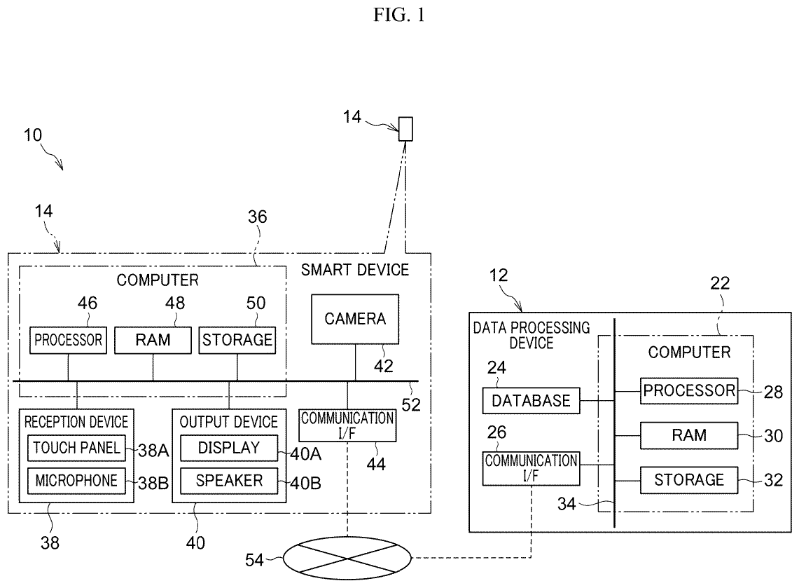

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

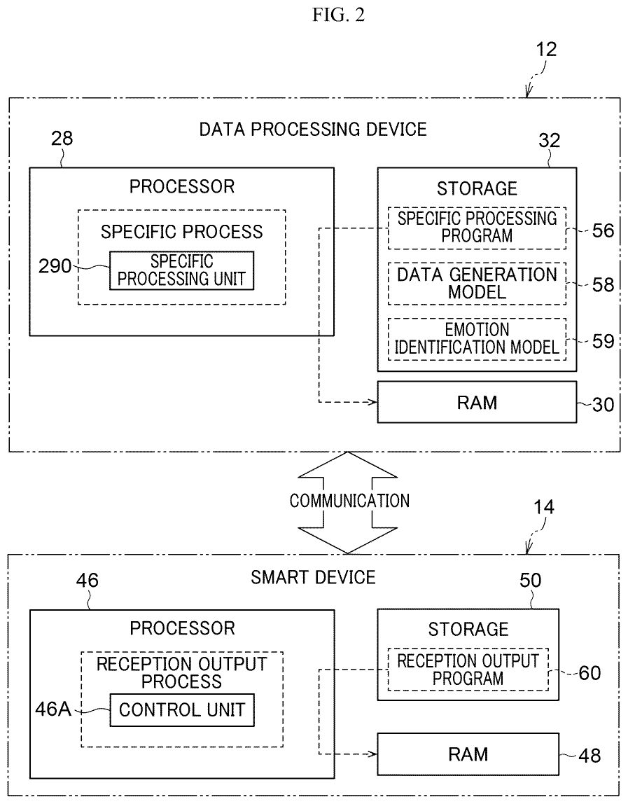

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional computer-implemented monitoring systems for elderly users typically rely on static questionnaires or manually designed rule sets to collect and evaluate health-related information. In many implementations, a fixed set of pre-authored questions is periodically presented to the user, and simple threshold-based logic is applied to raw numerical inputs, such as check boxes or severity scales. Such approaches suffer from several technical shortcomings from the perspective of computer technology.

First, conventional systems generally do not adapt the content or style of the questions to the evolving health history of the individual user. As a result, the interaction logic implemented by the computer remains static, even though a large amount of context data (for example, longitudinal symptom history and conversation logs) is stored in memory. This leads to low information density in each interaction, because the computer does not effectively exploit stored historical data when generating subsequent inputs for the user.

Second, where natural-language answers are accepted, conventional systems commonly store the raw text and, at most, apply shallow keyword matching. This forces later analysis to operate on unstructured text, which is computationally inefficient and error-prone. The lack of systematic conversion of natural-language responses into structured, machine-readable records reduces the accuracy and scalability of downstream analytics. In particular, it is difficult for the processor to reliably perform anomaly detection or trend analysis over long time periods, because the data remains poorly normalized and fragmented in storage.

Third, many existing systems do not integrate a generalized generative model into the end-to-end processing pipeline in a way that is tightly coupled with data storage and analysis components. Generative models, if used at all, are often invoked in an ad hoc manner, for example only to produce canned messages, without conditioning on structured historical data. This results in under-utilization of compute resources and model capabilities, and does not materially improve the technical behavior of the system, such as data throughput, robustness, or detection performance.

Fourth, conventional notification mechanisms typically apply simple rules over raw or loosely processed data, which can cause either excessive false positives (leading to notification fatigue for caregivers) or missed events. Because the computer does not consistently maintain and process structured symptom and severity information across multiple time steps, its internal decision logic for emergency detection is limited. This degrades the technical effectiveness of the monitoring system as an automated anomaly detection engine operating on a time-series of heterogeneous user inputs.

Accordingly, there is a need for an improved computer-implemented system that: (i) uses a generative AI model in a systematic manner to construct context-aware prompt sentences based on stored history information; (ii) converts free-form natural-language responses from an elderly user into structured data with explicit symptom, severity, and emotion attributes; (iii) performs statistical and anomaly detection processing over the structured data stored in a database; and (iv) controls automated notification transmission logic based on analysis results. Such a system should improve the overall technical performance of the health-monitoring pipeline, including more efficient utilization of stored data, more accurate and scalable analysis of user state over time, and more reliable triggering of emergency notifications by the processor.

The specific processing by the specific processing unit 290 of the data processing device 12 in Example 1 is realized by the following means.

The present invention provides a server comprising a processor and a communication interface, the processor being configured to construct, using stored history information including past health condition information and conversation history of an elderly user, a prompt sentence serving as input data to a generative AI model, the prompt sentence instructing the generative AI model to generate a natural language inquiry sentence for checking a current health condition of the elderly user, to transmit the inquiry sentence output from the generative AI model to a terminal device via the communication interface so that the inquiry sentence is presented as audio or text to the elderly user by the terminal device, to collect response information in natural language from the elderly user via the terminal device and convert the response information into structured data including at least symptom information, severity information, and emotion information by executing a natural language processing operation, to store the structured data in a database on a storage device in association with the inquiry sentence and time information by executing a database management operation, to read a plurality of pieces of the structured data from the database and execute statistical processing and anomaly detection processing to evaluate a change in the health condition of the elderly user and generate an analysis result indicating presence or absence of an abnormal state and a degree of urgency, and to control automatic transmission of a notification message via the communication interface to a registered notification destination including at least a family member or a caregiver when the analysis result satisfies a predetermined condition. This enables the computer system to automatically generate context-aware inquiry sentences using the generative AI model, to normalize free-form user responses into machine-readable structured records, to apply robust statistical and anomaly detection algorithms over time-series health data stored in the database, and to trigger technically reliable and timely emergency notifications based on the computed analysis results, thereby improving the technical performance and accuracy of computer-implemented health monitoring for elderly users.

The term “generative AI model” refers to an information processing model implemented in hardware and/or software that, in response to input data including a prompt sentence, generates new information expressed in a natural language, such as an inquiry sentence or notification message, based on learned statistical relationships in training data.

The term “prompt sentence” refers to a natural-language text sequence included in input data supplied to a generative AI model, the text sequence specifying at least a role, context, and generation instruction, and causing the generative AI model to generate a target output sentence such as an inquiry sentence or notification message.

The term “history information” refers to stored data representing past states and interactions of a user, including at least past health condition information, conversation history, and associated metadata such as timestamps, which is used as context for generating and analyzing subsequent communications.

The term “health condition information” refers to data representing physical and/or mental states of a user, including at least symptoms, severity levels, durations, and related contextual data that are relevant to health monitoring.

The term “conversation history” refers to stored records of past exchanges between a system and a user, including at least inquiry sentences presented to the user and corresponding responses provided by the user, represented as text and optionally associated with timestamps and additional attributes.

The term “natural language inquiry sentence” refers to a text string in a human language, generated by a generative AI model based on a prompt sentence, and configured to prompt a user to provide information about the user's health condition.

The term “terminal device” refers to an information processing device operated by or in proximity to a user, including at least a display unit, an input unit, and a communication unit, and configured to present inquiry sentences as audio or text and to capture user responses.

The term “response information” refers to user-provided data expressed in a natural language, obtained by the terminal device in reply to an inquiry sentence, and transmitted to a server for analysis and storage.

The term “structured data” refers to information represented in a machine-readable schema, including at least fields for symptom information, severity information, emotion information, and associated metadata, such that the information can be processed by statistical and anomaly detection algorithms.

The term “symptom information” refers to data elements identifying one or more health-related conditions or complaints reported by a user, such as pain, dizziness, or fatigue, encoded in a standardized or normalized form suitable for computerized analysis.

The term “severity information” refers to data elements representing an intensity or magnitude associated with a symptom, such as low, medium, or high severity, or a numerical score, derived from response information or rules and used for quantitative evaluation.

The term “emotion information” refers to data elements representing an emotional or affective state inferred from response information, such as anxiety, sadness, or calmness, encoded in a form suitable for computational analysis.

The term “database” refers to an organized collection of data stored on a storage device, managed by database management software, and structured to allow insertion, retrieval, update, and deletion of records including structured data associated with inquiry sentences and time information.

The term “database management operation” refers to a processing sequence executed by a processor to interact with a database, including at least creating, reading, updating, and deleting records, and associating structured data with inquiry sentences and time information.

The term “time information” refers to data indicating a temporal attribute associated with a record, such as a timestamp representing a date and time when an inquiry sentence was presented or a response was received.

The term “statistical processing” refers to computational operations applied to a set of structured data, including at least aggregation, averaging, frequency counting, and computation of temporal trends, performed to evaluate changes in health condition over time.

The term “anomaly detection processing” refers to computational operations that compare structured data to reference values or patterns, including rule-based or model-based techniques, to determine whether data points or sequences deviate from normal ranges or expected behavior.

The term “analysis result” refers to data generated by statistical processing and anomaly detection processing, indicating at least whether an abnormal state is present and, when present, a degree of urgency or severity associated with the state.

The term “abnormal state” refers to a condition determined by the system, based on structured data and analysis, in which a user's health condition deviates from a predefined normal or baseline range according to one or more rules or models.

The term “notification message” refers to information generated by the system for transmission to a notification destination, including at least a natural-language explanation of a detected abnormal state or change in health condition, and optionally recommended actions.

The term “notification destination” refers to one or more registered recipients, including at least a family member or caregiver associated with a user, to which a notification message is transmitted when a predetermined condition is satisfied.

The term “communication interface” refers to a hardware and/or software component configured to send and receive data over a communication network between the server and external devices, including at least terminal devices and notification destinations.

The term “summary text” refers to a natural-language representation generated from history information, including condensed descriptions of past symptoms and events, and used as part of input data to a generative AI model to improve context-aware generation of sentences.

The term “analysis summary information” refers to data representing a condensed explanation of changes in a user's health condition over a period, derived from analysis results and structured data, and suitable for inclusion in a notification message.

In one embodiment, a server manages end-to-end health monitoring for an elderly user by coordinating a generative AI model, a database system, and one or more terminal devices connected via a communication network. The server executes a computer program on a processor such as a multi-core central processing unit, and optionally accesses a graphics processing unit or tensor processing accelerator when executing neural-network inference.

The server runs an operating system such as a general-purpose server operating system, and hosts application software implemented, for example, in a programming language with libraries for natural language processing and data analysis. The server is connected, via a network interface, to a storage device that stores a relational database, and to an external communication service for notification delivery.

The server stores in the database history information representing past health condition information and conversation history of a user. The history information is stored as records in one or more tables, each record including a user identifier, a timestamp, an inquiry sentence previously presented to the user, and a response sentence received from the user. The server additionally stores structured data derived from the responses, including symptom information, severity information, and emotion information. The database may be implemented on a relational database management system, and the server may access the database via a database driver or an object-relational mapping library.

The server executes a generative AI model implemented as a neural network, for example a transformer-based architecture consisting of multiple self-attention layers, feedforward layers, and embedding layers. The generative AI model has been trained offline on a large corpus of natural language text using a supervised or semi-supervised learning procedure. The training procedure uses an objective function such as cross-entropy loss between predicted token distributions and ground-truth tokens, and updates weight parameters by backpropagation and stochastic gradient descent or a variant thereof. The model is hosted either locally on the server or on a separate inference service accessible via an application programming interface.

The server constructs, in memory, input data for the generative AI model including a prompt sentence and, in some embodiments, a summary text derived from the user's history information. The prompt sentence represents a sequence of tokens specifying at least a role for the model, a context for the elderly user, and an instruction to generate a natural language inquiry sentence. For example, the server may construct a system-level prompt such as:
“You are a generative AI model that creates one polite, concise daily health-check prompt sentence for an elderly user. Use simple language. Refer to the past few days'symptoms and ask about the current condition.”

The server then constructs a user-level prompt containing a summary of recent reports, such as:

“Recent reports:
Day—3: mild fatigue, no headache
Day—2: slight headache in the evening
Day—1: slight headache and light dizziness in the morning.

Generate exactly one prompt sentence to ask the user this morning about how they feel today.”

The generative AI model receives the concatenation of these prompt sentences as input, converts the tokens to continuous vector representations via an embedding matrix, propagates the vectors through self-attention layers that compute context-dependent representations, and outputs a probability distribution over possible next tokens at each position. The server samples or decodes tokens according to a decoding algorithm such as greedy decoding or top-k sampling, thereby obtaining a natural language inquiry sentence. An example of an output inquiry sentence is:
“Good morning. You mentioned some headaches and a little dizziness in the past few days; how are you feeling today, and do you still have any of those symptoms?”

The server uses this generation mechanism to adaptively shape the content of each inquiry sentence based on history information stored in the database. By structurally conditioning the prompt sentence on machine-readable summaries of past symptom and severity data, the server causes the generative AI model to focus its attention on medically relevant aspects, which improves the information density and relevancy of the resulting inquiry sentence. This improves computational efficiency, because fewer interactions are needed to obtain sufficiently rich health information, and reduces redundant or irrelevant questions.

The terminal operates as a user interface device, such as a smartphone, tablet, or dedicated monitoring device. The terminal comprises a display unit, an audio output unit, an input unit including a touchscreen and optionally a microphone, and a communication unit. The terminal receives an inquiry sentence from the server via a communication protocol such as Hypertext Transfer Protocol Secure, stores the text content in local memory, and renders the inquiry sentence in a user interface. The terminal may also convert the text inquiry sentence to speech by using a text-to-speech engine installed on the terminal, and output audio to the user.

The user reads or listens to the inquiry sentence on the terminal and then provides a response in natural language, for example by typing on a touchscreen keyboard or by speaking into the microphone. The terminal converts any speech input to text using a speech recognition engine, and represents the response as a character sequence. The terminal then transmits the response information as a text payload to the server, together with metadata such as a timestamp, a prompt identifier, and a user identifier.

The server receives the response information and performs natural language processing using one or more software libraries. The server may use a pipeline including tokenization, part-of-speech tagging, syntactic parsing, and named-entity recognition. The server defines domain-specific entities representing symptoms, intensifiers, durations, and emotional cues. The server maps lexical expressions such as “slight headache” or “very dizzy” to symptom codes and severity values by applying a combination of rule-based mapping tables and classification models. For example, the server may define a dictionary that associates words such as “slight”, “mild”, and “a bit” with low severity scores, and words such as “severe”, “strong”, and “intense” with high severity scores. The server may also deploy a classifier model that receives token embeddings of the response sentence as input and outputs a multi-label vector indicating the presence of specific symptom categories.

The server converts the analysis result into structured data with explicit fields for symptom information, severity information, emotion information, and associated timestamps. The server stores this structured data into the database as one or more records, using a fixed schema. For example, the server may store, for each report, a list of symptom identifiers, a numeric severity score normalized to a predefined range, and an emotion vector indicating probabilities of emotional states. By structuring data in this manner, the server enables efficient indexing and querying, reducing computational complexity for downstream statistical processing.

The server periodically retrieves a plurality of structured records from the database and performs statistical processing and anomaly detection. The server loads the data into an in-memory data structure suitable for bulk operations, and computes quantities such as symptom occurrence frequencies within a sliding time window, moving averages of severity scores, and variance measures relative to a baseline. The server defines baseline values for each symptom and severity dimension based on historical data or population-level parameters stored in configuration.

The server applies anomaly detection algorithms to the processed data. In one embodiment, the server computes a z-score for each symptom dimension, indicating how many standard deviations the current frequency or severity deviates from the baseline. The server classifies states whose z-score exceeds a threshold as abnormal. In another embodiment, the server trains an unsupervised anomaly detection model, such as an isolation forest or an autoencoder neural network, using past structured data as training input. The server feeds current structured data to the model, and obtains an anomaly score representing how unusual the pattern is compared to typical historical patterns. The server then compares the anomaly score to a predetermined threshold to determine whether an abnormal state exists.

The server determines, based on analysis results, a degree of urgency for each detected abnormal state. The server defines rule sets that combine anomaly scores, symptom types, and severity values. For example, the server may specify that repeated high-severity headache combined with dizziness over three consecutive days constitutes a high-urgency state, while occasional low-severity fatigue constitutes a low-urgency state. By codifying such rule sets in software, the server implements a deterministic decision logic that can be audited and tuned.

The server controls automatic transmission of notification messages based on the determined degree of urgency. When the server determines that an abnormal state satisfies a predetermined condition for notification, the server constructs a notification message. In one embodiment, the server uses templates containing placeholders for symptom names, severity levels, and time ranges, and fills the placeholders with values computed from structured data. In another embodiment, the server uses the generative AI model to create a notification message in natural language that explains the abnormal state. The server then sends the notification message to a notification destination via a communication service, such as an SMS gateway or an email service. The server records the notification transmission status in the database, so that subsequent analysis can take into account whether caregivers have been alerted.

The server improves computer technology in several ways. First, the server reduces storage and processing inefficiency by converting free-form natural language input into structured data aligned with a well-defined schema. This enables indexed queries and vectorized operations on symptoms and severity values, which reduces query response times and computational overhead compared to processing unstructured text each time. Second, the server improves the accuracy of anomaly detection by integrating learned models with deterministic rules that operate on normalized symptom vectors, reducing false positives that would result from keyword-based matching.

Third, the server improves interaction efficiency by using the generative AI model to generate context-aware inquiry sentences that are conditioned on summary text derived from historical data. Because the server includes the summary text in the prompt sentence, the generative AI model focuses on recent symptom patterns and generates questions that specifically probe changes that matter for anomaly detection. This reduces the number of interactions required to gather relevant data and reduces network and processing load on the server and the terminal.

Fourth, the server improves robustness and scalability of the monitoring pipeline by maintaining a clear separation between natural language processing, data structuring, statistical analysis, and notification control. Each module uses explicit data structures and algorithms, allowing optimization of each step. For example, the server may cache intermediate summary texts to avoid recomputing them for each prompt generation, or may batch anomaly detection computations across multiple users to exploit vectorized operations.

The server further distinguishes the described processing from mere automation of human tasks by implementing non-intuitive, computer-specific steps. For instance, the server uses continuous symptom embeddings derived from the neural network's internal states as features for anomaly detection models. These embeddings capture similarity relationships between different linguistic expressions of symptoms in a high-dimensional space, enabling the system to recognize equivalent patterns that would be difficult to codify in hand-crafted rules. The server also applies dimensionality reduction techniques to these embeddings to reduce computational cost in subsequent detection stages. These operations are inherently computational and rely on properties of high-dimensional vector spaces, rather than simply replicating a human caregiver's mental process.

In another embodiment, the server uses a variant of the transformer architecture with attention heads specialized for health-domain keywords. During training, the model incorporates domain-specific vocabularies and uses data augmentation methods such as synonym replacement and paraphrasing to increase robustness to variation in user phrasing. The training algorithm adjusts attention weights so that symptom-related tokens receive higher attention scores, resulting in improved generation precision and improved extraction of symptom information from user responses. This targeted attention mechanism enhances compute efficiency by concentrating representational capacity on medically relevant segments of the input.

The terminal may implement alternative user interface modalities. In one variation, the terminal uses only text input and output, while in another variation, the terminal uses both text and synthesized speech to accommodate users with visual impairments. The terminal may locally cache the last generated inquiry sentence and display it even if connectivity is momentarily unavailable, and the server may reconcile cached responses when connectivity is restored. Such variations still rely on the same underlying data flow between the server, the database, the generative AI model, and the anomaly detection modules.

In yet another embodiment, the server executes the generative AI model on a dedicated accelerator, such as a graphics or tensor processing unit, and executes statistical and anomaly detection processing on the central processing unit. The server manages queues of generation and analysis tasks, and balances workloads between processing units. By doing so, the server reduces latency in generating inquiry sentences and processing responses, enabling near real-time feedback. This architecture provides a technical improvement in throughput over conventional systems that rely on a single processor for all tasks.

The user interacts with the system by receiving inquiry sentences on the terminal and providing honest responses. The system does not require the user to understand any technical aspects of the processing, and all complex analysis operations are handled by the server. However, the resulting improvement in detection speed and accuracy manifests in the real world as earlier identification of potentially dangerous health changes, enabling timely intervention.

Multiple variations in generative AI model type, database implementation, and anomaly detection algorithm may be used without departing from the scope of the invention, so long as the server constructs prompt sentences based on history information, generates inquiry sentences using a generative AI model, converts responses into structured data, evaluates changes in health condition using statistical and anomaly detection techniques, and controls automated notification transmission based on analysis results. By this integrated design focused on internal data structures and algorithms, the system produces technical effects in terms of improved computational efficiency, accuracy of anomaly detection, and reliability of automated monitoring, beyond simple automation of human evaluation tasks.

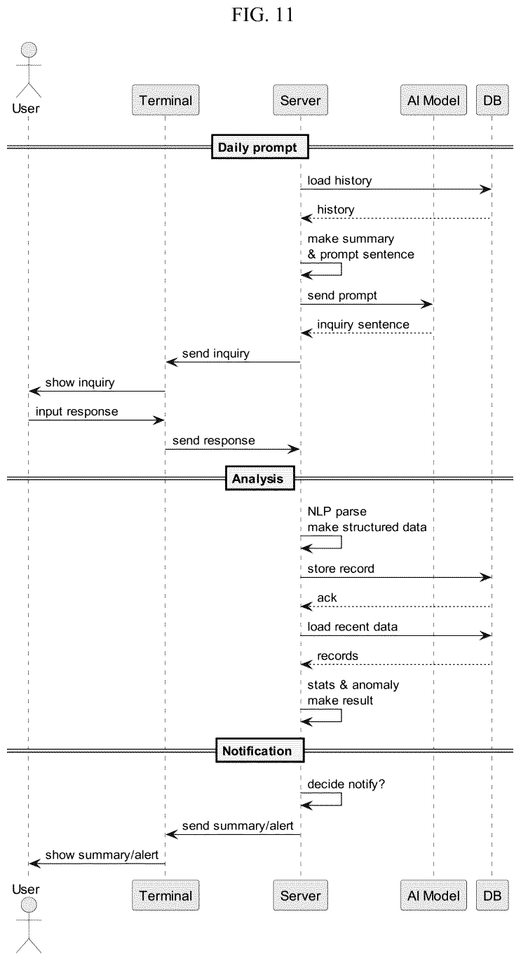

The following describes the processing flow using FIG. 11.

### Step 1:

The server reads history information for a target user from a database.

The server receives, as input, a user identifier and a time range. Based on this input, the server executes database queries to load past health condition records and conversation history within the specified time range. The server converts the query results into in-memory data structures, such as lists of records or tables of timestamps, inquiry sentences, response sentences, symptom codes, severity scores, and emotion scores. The server outputs a structured history dataset that will be used as context for subsequent processing.

### Step 2:

The server generates a summary text from the history dataset.

The server receives, as input, the structured history dataset containing multiple records of symptoms and severities over time. The server groups records by day and symptom type, counts how many times each symptom has appeared, and computes statistics such as maximum severity per day. Using these aggregated values, the server constructs a concise natural language summary, for example: “Recent reports: Day—3: mild fatigue, no headache; Day—2: slight headache in the evening; Day—1: slight headache and light dizziness in the morning.” The server outputs the summary text as a character string.

### Step 3:

The server constructs a prompt sentence for a generative AI model.

The server receives, as input, the summary text and configuration parameters specifying the desired style and length of an inquiry sentence. The server concatenates a role description, an instruction, and the summary text to form a multi-part prompt sentence, for example:
“You are a generative AI model that creates one polite, concise daily health-check prompt sentence for an elderly user. Use simple language. Refer to the past few days'symptoms and ask about the current condition.

Recent reports: . . .

Generate exactly one prompt sentence to ask the user this morning about how they feel today.”

The server encodes this prompt sentence in a format required by the generative AI model interface and outputs the formatted prompt as input data for the model.

### Step 4:

The server generates a natural language inquiry sentence using the generative AI model.

The server receives, as input, the formatted prompt sentence. The server sends the prompt to the generative AI model and receives token-level probability distributions for the next output tokens. The server performs a decoding algorithm, such as greedy decoding, to select at each step the most probable token, and continues until an end-of-sentence condition is met. The server concatenates the selected tokens to form a natural language inquiry sentence, for example: “Good morning. You mentioned some headaches and a little dizziness in the past few days; how are you feeling today, and do you still have any of those symptoms?” The server outputs this inquiry sentence as a text string.

### Step 5:

The server transmits the inquiry sentence to the terminal.

The server receives, as input, the inquiry sentence and a terminal identifier associated with the user. The server wraps the inquiry sentence, together with metadata such as a prompt identifier and timestamp, into a communication message and sends the message to the terminal via a network protocol. The server outputs a delivery status indicating success or failure of the transmission.

### Step 6:

The terminal presents the inquiry sentence to the user.

The terminal receives, as input, the communication message containing the inquiry sentence and metadata. The terminal parses the message, extracts the inquiry sentence text, and renders it on a display, and optionally converts it to audio using a text-to-speech engine. The terminal presents the text and/or audio to the user, and waits for input. The terminal outputs a visual and/or auditory presentation that the user can perceive.

### Step 7:

The user provides a natural language response through the terminal.

The user receives, as input, the presented inquiry sentence asking about the current health condition. The user cognitively interprets the question and generates a response, such as “My headache is worse today, and I feel a bit dizzy.” The user enters this response using the input interface of the terminal, such as a virtual keyboard or a microphone. The user outputs a natural language response in textual or spoken form to the terminal.

### Step 8:

The terminal converts the user's response into text and sends it to the server.

The terminal receives, as input, the response provided by the user, either as typed text or as an audio signal. When the input is audio, the terminal executes a speech recognition process to convert the audio waveform into a text string. The terminal then attaches metadata including the prompt identifier, user identifier, and a timestamp, and generates a structured message. The terminal transmits this message to the server via the network. The terminal outputs a text-based response message to the server.

### Step 9:

The server performs natural language processing on the response text.

The server receives, as input, the response text and associated metadata. The server tokenizes the text into words or subword units, assigns part-of-speech tags, and identifies syntactic dependencies. The server applies domain-specific rules and models to detect mentions of symptoms, modifiers indicating severity, temporal expressions, and emotional phrases. For example, the server detects “headache” as a symptom and “worse” as indicating increased severity, and detects “a bit dizzy” as a separate symptom with low severity. The server outputs an intermediate representation that includes identified symptom expressions, severity indicators, and emotion cues.

### Step 10:

The server converts the intermediate representation into structured data.

The server receives, as input, the intermediate representation of the response. The server maps each detected symptom phrase to a standardized symptom code, and maps intensity words to numeric severity levels within a predefined scale. The server may compute an emotion score vector based on the presence of specific emotional keywords or by using a classifier model. The server packages this information into a structured record containing fields for user identifier, timestamp, symptom codes, severity scores, and emotion scores. The server outputs a structured data record suitable for storage in the database.

### Step 11:

The server stores the structured data and updates the history in the database.

The server receives, as input, the structured data record and any associated raw text. The server executes a database insertion operation, adding a new row to a health-report table, and links the record to the corresponding inquiry sentence via the prompt identifier. The server may also update summary tables that maintain rolling statistics, such as counts of each symptom in the last seven days. The server outputs updated database contents and an acknowledgment indicating successful storage.

### Step 12:

The server performs statistical processing on accumulated structured data.

The server receives, as input, a set of structured data records for the user over a specified time window. The server aggregates the data by symptom code and time interval, computes frequencies of each symptom, calculates moving averages of severity scores, and determines maximum and minimum values for each metric. For example, the server may compute that “headache” has appeared on four of the last seven days with average severity of 0.7 on a 0-1 scale. The server outputs statistical summaries for each relevant symptom and time period.

### Step 13:

The server executes anomaly detection on the statistical summaries.

The server receives, as input, the statistical summaries and baseline reference values for each symptom. The server computes deviation measures such as differences between current and baseline frequencies, and z-scores representing standardized deviations. The server then applies decision rules or model-based anomaly detectors to classify whether each symptom pattern is normal or abnormal. For instance, if the frequency and severity of headache exceed thresholds for several consecutive days, the server marks this pattern as abnormal. The server outputs an analysis result that includes flags for abnormal symptoms and an overall urgency level.

### Step 14:

The server determines whether to trigger a notification based on the analysis result.

The server receives, as input, the analysis result containing abnormality flags and urgency information. The server compares the urgency level and specific symptom combinations against predefined notification criteria. If the analysis result satisfies a condition for notification, such as “severe headache and dizziness reported on three consecutive days,” the server decides to generate a notification. If the criteria are not met, the server decides not to send a notification. The server outputs a notification decision indicating whether a notification should be generated and, if so, the intended notification destinations.

### Step 15:

The server generates a notification message for a notification destination.

The server receives, as input, the notification decision, the structured data for the relevant time window, and optionally the analysis summaries. The server either fills a message template with symptom names, durations, and severity levels, or constructs a prompt sentence and calls the generative AI model to create a natural language explanation. For example, the server may generate a message such as: “Your mother has reported worsening headaches and dizziness over the last three days. A medical consultation is strongly recommended.” The server outputs a complete notification message suitable for transmission.

### Step 16:

The server transmits the notification message via an external communication service.

The server receives, as input, the notification message and contact information for the notification destination, such as a telephone number or email address. The server formats an API request for an external communication service and sends the request over the network. The external service attempts delivery of the message. The server receives a delivery status response and stores this status in the database linked to the notification record. The server outputs a final status indicating whether the notification was successfully delivered.

### Step 17:

The terminal optionally displays a summary of the analysis result to the user.

The terminal receives, as input, a status message from the server containing a brief summary of recent health monitoring, such as “Frequent headaches detected; please consider resting and consulting a doctor.” The terminal parses the message and presents the summary on the display in a dedicated status screen or notification area. The terminal may highlight abnormal conditions with visual indicators. The terminal outputs this visual feedback so that the user can understand the current evaluation of their health condition.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional health monitoring systems for elderly users primarily rely on fixed questionnaires and simple rule-based analysis executed on general-purpose computing platforms. Such systems exhibit several technical limitations. First, because question content is static and not tailored to the user's current and historical state, the system generates redundant or irrelevant prompts, which unnecessarily consume network bandwidth, processing resources, and storage capacity across terminals, servers, and cloud infrastructures. Second, conventional systems process user responses as unstructured text logs with minimal linguistic understanding, leading to inefficient use of computing resources for downstream analysis and limiting the ability to automatically detect subtle risk patterns over time. Third, notification mechanisms are often triggered by simple threshold checks on single data points, rather than by coordinated processing of time-series health data and adaptive interaction policies, resulting in either excessive false alarms that burden communication channels and server resources, or missed detection of critical events.

Furthermore, existing architectures typically separate the interaction layer at the user terminal from the analytical layer at the server in a rigid manner, without a feedback loop that uses server-side analysis results to dynamically reconfigure the generation of subsequent questions or guidance. As a result, the system cannot optimize the behavior of generative AI models or natural language processing pipelines in response to accumulated health state information. This leads to suboptimal allocation of computation between the user terminal and the server, unnecessary repeated processing of low-value data, and increased latency in detecting and responding to abnormal health conditions.

Accordingly, there is a need for a computer-implemented health monitoring system that technically improves the way processors manage dialog generation, natural language analysis, abnormality determination, and notification control. In particular, there is a need for a system in which a processor coordinates input prompt sentences to a generative AI model, server-side natural language processing, and cloud-based storage and analysis of structured health state information, so that question generation, risk calculation, and alerting are adaptively and efficiently controlled. Such a system should reduce redundant computation and communication, improve the quality and structure of the data being processed, and enhance the reliability and timeliness of abnormality detection by using the computing resources of terminals, servers, and cloud databases in a more effective manner.

The specific processing by the specific processing unit 290 of the data processing device 12 in Application Example 1 is realized by the following means.

The present invention provides a server comprising a processor and a memory storing instructions which, when executed by the processor, cause the server to cooperate with a user terminal and a cloud-based storage system by: generating and transmitting, to a generative AI model operating on an information processing infrastructure, prompt sentences and control prompt sentences configured to cause the generative AI model to output personalized question sentences and feedback sentences for an elderly user; receiving, from the user terminal via a communication network, answer text data obtained by converting response audio information of the elderly user into text, and performing natural language processing on the answer text data to extract structured health state information including symptom information, emotional state information, and severity information; storing the extracted health state information, in association with user identifying information and time information, in a database in the cloud-based storage system; periodically computing, based on multiple pieces of the stored health state information across different time points, a health risk index for the elderly user and executing an abnormality determination process to determine whether the health risk index satisfies a predetermined abnormality condition; when the abnormality condition is satisfied, generating notification messages including latest health state information and abnormality content and automatically transmitting the notification messages to contact destinations via at least one communication service; and dynamically updating, based on the health state information and the health risk index, subsequent prompt sentences used for generating future question sentences and guidance for the elderly user so that dialog content and frequency are adaptively controlled. This enables technical improvements in computer operation by reducing unnecessary data transmission and processing through adaptive, state-aware prompt generation, by transforming unstructured response data into structured health state information that is efficiently analyzed over time, by optimizing server-side and terminal-side resource usage through coordinated generative AI and natural language processing, and by enhancing the accuracy and timeliness of abnormality detection and notification within a distributed computing environment.

The term “system” refers to an arrangement of one or more computing devices, communication networks, and storage resources that cooperate to execute the functions described in the claims.

The term “processor” refers to any hardware execution unit, such as a central processing unit or a microcontroller, that executes machine-readable instructions to perform operations on data.

The term “memory” refers to any non-transitory computer-readable storage medium that stores instructions or data for use by a processor.

The term “user terminal” refers to any portable or stationary computing device, including a mobile communication device or a general-purpose computer, that is operated by an elderly user to input and receive information.

The term “elderly user” refers to a human user belonging to an age group for which regular health monitoring is desired due to age-related health risks.

The term “attribute information” refers to data representing characteristics of the elderly user, including at least one of age information, gender information, medical history information, medication information, lifestyle information, or living environment information.

The term “health-check purpose for a current day” refers to information indicating a monitoring objective for a specific time period, including at least one of screening for new symptoms, tracking known conditions, or assessing daily well-being.

The term “prompt sentence” refers to a natural-language text or structured text that is provided as input to a generative AI model to condition or guide generation of output text.

The term “guidance prompt sentence” refers to a prompt sentence configured to cause a generative AI model to generate feedback sentences including lifestyle precautions or medical consultation recommendations.

The term “control prompt sentence” refers to a prompt sentence configured to instruct a generative AI model regarding a continuous monitoring policy and at least one of content and frequency of question sentences to be generated in subsequent sessions.

The term “generation instruction information” refers to data indicating that a prompt sentence is to be input to a generative AI model in order to generate one or more question sentences or feedback sentences.

The term “generative AI model” refers to a machine learning model that generates text data, including question sentences or feedback sentences, based on input text such as a prompt sentence.

The term “information processing infrastructure” refers to one or more computing resources, including servers and associated components, on which the generative AI model is executed and which are accessible via a communication network.

The term “question sentence” refers to text generated by a generative AI model that is configured to query an elderly user regarding the elderly user's health condition, feelings, symptoms, or related matters.

The term “feedback sentence” refers to text generated by a generative AI model that provides the elderly user with guidance, advice, lifestyle precautions, or medical consultation recommendations based on health-related information.

The term “communication network” refers to any wired or wireless communication facility, including local and wide area networks, that enables data exchange between a user terminal, a server, and an information processing infrastructure.

The term “display function” refers to a capability of a user terminal to visually present characters, images, or graphical user interfaces on a display device.

The term “voice output function” refers to a capability of a user terminal to output audio signals, including synthesized speech corresponding to text, through a sound reproduction device.

The term “response audio information” refers to audio data representing spoken responses of the elderly user to one or more question sentences.

The term “speech recognition processing” refers to processing that converts audio data, including response audio information, into corresponding text data using an automatic speech recognition algorithm.

The term “answer text data” refers to text data that represents the content of a response of the elderly user to a question sentence, obtained by converting audio information or by directly inputting text.

The term “temporary storing” refers to holding data in a memory or storage area for a period sufficient to perform subsequent processing, without requiring permanent retention.

The term “server” refers to one or more computing devices that receive data from the user terminal, execute natural language processing, perform health risk determination, manage access to a database, and control notification operations.

The term “natural language processing technique” refers to any algorithm or computational process that analyzes text in a human language to extract structured information, including at least tokenization, classification, entity extraction, or sentiment analysis.

The term “health state information” refers to structured data representing a health-related state of the elderly user, including at least one of symptom information, emotional state information, and severity information.

The term “symptom information” refers to data indicating a physical or mental sign, complaint, or condition reported or inferred from the elderly user's responses.

The term “emotional state information” refers to data representing an inferred or reported psychological or emotional condition of the elderly user, such as anxiety, sadness, or stability.

The term “severity information” refers to data indicating an intensity or seriousness level of a symptom or overall health condition of the elderly user.

The term “user identifier” refers to data that uniquely or distinctively identifies an elderly user within the system.

The term “time information” refers to data that indicates when a question was presented, when a response was received, or when analysis or storage operations were performed.

The term “database” refers to an organized collection of data stored in a storage device and managed by database management software to support structured storage and retrieval.

The term “cloud environment” refers to a computing environment in which storage and processing resources are provided by remote computing facilities accessible via a communication network.

The term “health risk index” refers to a value or set of values computed based on multiple pieces of health state information, representing a level of health risk for the elderly user.

The term “abnormality determination process” refers to a process in which a health risk index is evaluated against one or more conditions to determine whether an abnormal health state exists for the elderly user.

The term “abnormality determination condition” refers to at least one threshold or rule defining when a health risk index indicates an abnormal or potentially dangerous health state.

The term “contact information storage unit” refers to a storage component that holds data representing contact destinations, including addresses or identifiers for communication with family members or healthcare providers.

The term “contact information” refers to data specifying a destination for communication, including at least one of a telephone number, an electronic mail address, or a device identifier for push notifications.

The term “notification message” refers to text or structured data that conveys information regarding health state, abnormality content, or alerts to a contact destination.

The term “message generation processing” refers to processing that constructs a notification message or feedback content by combining templates with variable data, including health state information and abnormality content.

The term “short-range text communication service” refers to a communication service capable of transmitting short text messages over a public or private network.

The term “electronic mail service” refers to a communication service that enables transmission and reception of electronic mail messages over a communication network.

The term “notification delivery service” refers to a service that delivers alert or status messages, including push notifications, to one or more devices or applications associated with contact information.

The term “continuous monitoring policy” refers to a set of conditions and rules defining how frequently, under what circumstances, and with what content the system interacts with the elderly user to monitor health.

The term “session” refers to a period of interaction in which one or more question sentences are presented to the elderly user and corresponding responses are collected and processed.

The term “personalized question sentence” refers to a question sentence whose content is selected or generated based on at least one of attribute information, past health state information, symptom occurrence history, or a health risk index of the elderly user.

The term “symptom occurrence history” refers to data representing temporal patterns, counts, or sequences of symptoms previously extracted as part of health state information.

The term “subsequent question sentences” refers to question sentences that are generated and presented after one or more preceding sessions, taking into account accumulated health state information and control prompt sentences.

The term “lifestyle precaution” refers to advice or guidance suggesting specific behaviors or habits intended to reduce health risks for the elderly user.

The term “medical consultation recommendation” refers to guidance indicating that the elderly user should seek evaluation, diagnosis, or treatment from a healthcare professional or institution.

In one embodiment, a system includes a server, a terminal, and a user, where the user is an elderly person who interacts with the terminal to provide health-related information on a daily basis.

The server operates on one or more computing devices, such as rack-mounted computers or virtual machines, each having at least one central processing unit (CPU), a main memory, a network interface, and a non-transitory storage device. The server executes an operating system such as a general-purpose server operating system and an application stack including a web application framework, a natural language processing (NLP) library, and a database client library. The server further accesses a cloud-based database system, such as a relational database or a document-oriented database, and an external or internal generative AI model execution environment.

The terminal operates as a mobile communication device or general-purpose client computer, such as a smartphone or tablet, including a processor, a display, a microphone, a speaker, a wireless communication interface, and local storage. The terminal executes a health-monitoring application program that communicates with the server and with a generative AI model via a communication network such as a cellular network and a packet-switched network.

The user interacts with the terminal by viewing and listening to question sentences presented by the terminal, and by inputting responses in the form of speech or text. The user does not need to manually select question content; instead, the question content is formed by cooperative processing of the server and the generative AI model based on accumulated health state information stored in the database.

In the described embodiment, the server uses a generative AI model that is implemented as a neural network having a transformer architecture. The generative AI model includes an encoder-decoder or decoder-only structure composed of multiple layers of self-attention units, feedforward networks, and normalization modules. The generative AI model receives as input a sequence of tokens representing a prompt sentence and outputs a sequence of tokens representing generated text. The tokens are numerical representations, such as integer indices mapped to embeddings; the embeddings are vectors in a continuous space. The model is trained by a supervised or self-supervised learning method on large-scale text data, using an objective function such as cross-entropy loss comparing predicted token distributions with ground truth tokens. During training, the model adjusts its internal weight parameters using gradient descent or a variant thereof, such as Adam optimization, based on a backpropagation algorithm that computes gradients of the loss with respect to each weight.

The server uses the generative AI model execution environment in a way that improves computer operation beyond simple human task automation. The server constructs specialized prompt sentences that include machine-readable markers and structured health-related context, such as summarized symptom history and computed risk scores, encoded into natural-language segments. Because the server adapts the prompt sentences to the current state of the database and the outcome of the abnormality determination logic, the generative AI model generates question sentences that are constrained and focused on technically relevant health dimensions. This reduces the volume and redundancy of generated text, lowers the number of network round trips between the server and the terminal, and decreases processing load on both the terminal and the server compared to conventional static questionnaires.

The server uses NLP modules based on transformer or recurrent neural network models for analysis of user responses. In one embodiment, the server employs a classifier model that receives tokenized answer text data and outputs probability distributions over symptom categories, emotional states, and severity levels. The classifier is trained using labeled training data in which each text example has one or more assigned labels corresponding to medically relevant symptom categories and severity levels. The server uses a multi-label classification loss such as binary cross-entropy and adjusts model parameters by gradient-based optimization. In another embodiment, the server uses a sequence tagging model that assigns labels to each token, thereby extracting symptom terms and modifiers. This processing converts unstructured answer text data into structured health state information, enabling efficient indexing, querying, and time-series analysis in the database.

The server employs specific data structures to represent health state information. For example, the server stores, in the database, session records containing fields such as a user identifier, a session identifier, a date-time stamp, and an overall risk score. The server stores answer records containing fields such as a question identifier, raw answer text, and language metadata. The server further stores symptom records containing fields such as a symptom type code, a severity level, an emotional state code, and a confidence score output by the classifier. These structures allow the server to compute a health risk index using numeric aggregation and pattern detection algorithms, such as moving averages, trend analysis, or rule-based combinations of severity levels and symptom types.

The server calculates the health risk index using an algorithm that differs from conventional fixed-threshold checks. The server uses time-windowed evaluation, where the server retrieves multiple health state entries for a defined past period, such as several days or weeks, and applies a function that weights recent entries more heavily than older entries. The server may use an exponential moving average or a logistic regression model that combines symptom severity features, frequency of certain symptom types, and emotional state features to output a risk score. This computation is designed to be efficient in terms of database queries and CPU usage by relying on pre-aggregated symptom records and indexed fields, which reduces the number of full-text scans and complex joins.

The terminal performs signal processing and user-interface control. The terminal captures audio signals using an analog-to-digital converter and stores digital audio samples in a buffer. The terminal then sends these audio samples to a speech recognition engine implemented as a neural network, such as a convolutional or recurrent architecture trained on acoustic features like Mel-frequency cepstral coefficients. The speech recognition engine outputs a sequence of text tokens representing the user's spoken response. The terminal converts these tokens into answer text data, associates them with identifiers for the corresponding question sentences, and transmits the answer text data to the server.

In an alternative embodiment, the terminal executes part of the NLP processing locally using a lightweight model, thereby performing on-device preliminary classification of answers. The terminal can compute coarse symptom indicators, such as flags for “pain”, “dizziness”, or “no appetite”, and attach these flags to the payload sent to the server. This reduces server computation for trivial or clearly normal cases and enables faster local feedback to the user, while still allowing detailed server-side analysis when required.

The user receives question sentences and feedback sentences that are both adapted to the user's individual health state. For example, the server may generate a prompt sentence:
“Please generate three short, polite questions in English to check the daily health condition of an elderly person with a recent history of dizziness and reduced appetite. Focus on any change in dizziness, ability to walk, and food intake since yesterday.”

The generative AI model receives this prompt sentence and outputs question sentences such as:
“How is your dizziness today compared to yesterday?”
“Do you feel any difficulty when walking or standing up?”
“How much did you eat at your last meal?”

In another example, the server may generate a guidance prompt sentence:
“Please generate a short, empathetic advice message in English for an elderly person who reported mild dizziness and leg pain, focusing on safe behavior at home and when to contact a doctor.”

The generative AI model returns a feedback sentence such as:
“If you feel dizzy, please sit or lie down immediately and avoid walking alone on stairs. If the dizziness or leg pain becomes stronger or does not improve, please contact your doctor or a medical facility as soon as possible.”

The server transmits these feedback sentences to the terminal, and the terminal displays them on the screen or reads them aloud using a text-to-speech engine.

By coordinating prompt sentence generation, generative AI model output, and server-side risk analysis, the system improves the underlying computer technology. The server reduces communication load by avoiding generic, repetitive question sets and instead requesting only those question sentences that add diagnostic value based on the stored time-series health state data. The server optimizes database access patterns by storing structured symptom records, which permits index-based retrieval and aggregated computations, thereby lowering latency and CPU usage compared to systems that only store raw text logs. The server also reduces error rates in abnormality detection, because the risk index calculation uses multiple features derived from NLP outputs, rather than single, static thresholds applied to raw answers.

The system uses non-conventional control of the generative AI model through control prompt sentences that encode a continuous monitoring policy. Instead of human operators manually changing questionnaire content, the server dynamically updates the control prompt sentences according to algorithmic rules based on health state information and risk index. For instance, when the risk index remains low over multiple sessions, the server decreases the frequency of questions by including instructions such as “ask only one question focusing on overall condition,” while when the risk index rises, the server adds instructions such as “ask detailed follow-up questions about breathing, chest pain, and confusion.” This control loop modifies the behavior of the generative AI model in a manner that is not simply replicating human questionnaire design; it exploits the model's ability to follow high-level, machine-generated policies encoded in prompt sentences.

The server applies these control prompt sentences in a consistent, machine-executable way. The server constructs the control prompt sentences by combining machine-readable markers, such as “[RISK_LEVEL=HIGH]” or “[SYMPTOM_TREND: dizziness: increasing]”, with natural-language instructions. The generative AI model, having been trained to interpret such markers, adjusts its output distribution accordingly. This technique results in more precise and constrained text generation, thereby improving computational efficiency and enabling deterministic adaptation of dialog content.

The system achieves technical effects that include improved processing speed, reduced communication overhead, improved classification accuracy of health states, and more efficient data management. The improved processing speed arises from the use of structured health state information and pre-aggregated risk indices, which allow the server to execute abnormality determination without re-processing all historical raw text. The communication overhead is reduced by sending concise, context-rich prompt sentences and by limiting question generation and answer transmission to what is necessary for updated risk calculation. The classification accuracy is improved by training and employing specialized NLP models that are tuned to health-related symptom extraction and emotional state recognition. The data management is improved by defining specific database schemas and indexing strategies for health state information, which support scalable and reliable operations in a cloud environment.

In another embodiment, the server may employ different neural network architectures or data processing pipelines. For example, the server may use a bidirectional encoder to generate contextual embeddings of answer text data and then apply a feedforward classifier to output symptom probabilities. The server may also use dimensionality reduction techniques such as principal component analysis on features derived from symptom occurrence history to simplify the risk index computation. The generative AI model may vary in size, number of layers, or vocabulary, and yet the system maintains the control mechanism via prompt sentences and control prompt sentences.

In yet another embodiment, the server may incorporate user feedback regarding incorrect or incomplete question sentences or feedback sentences. The server can store such feedback, retrain or fine-tune portions of the NLP or classification models using supervised learning with updated labels, and adjust the internal thresholds for abnormality determination. This creates a continuous improvement loop that further refines the technical performance of the system over time.

Through these configurations, the server, the terminal, and the user form a distributed computing system in which each component executes defined roles: the terminal performs acquisition of audio signals, local conversion, and interface control; the server performs structured data extraction, risk computation, and notification control; and the generative AI model executes guided text generation under technically constrained prompt sentences. This arrangement enables concrete technical improvements in how computing devices query, process, and act upon health-related information, going beyond abstract data processing and providing a specific improvement to the functioning of the computers and networks themselves.

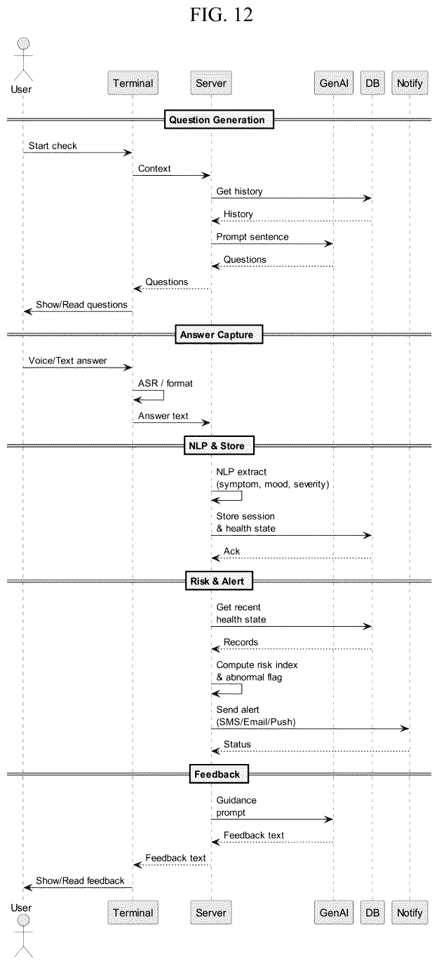

The following describes the processing flow using FIG. 12.

### Step 1:

Terminal initializes monitoring context and user profile.

Terminal receives as input internal configuration data, a current timestamp from an internal clock, and user profile data stored in local storage (for example, age, known conditions, medication information). Terminal reads these values from a local database, decodes them into in-memory objects, and computes a monitoring context that includes the current date, time-of-day, and recent interaction status. Terminal outputs a context data structure containing user attribute information, a session identifier, and monitoring objectives (for example, “daily check” or “follow-up on dizziness”).

### Step 2:

Server constructs a prompt sentence for a generative AI model.

Server receives as input the context data structure from the terminal via a communication network. Server parses the context, extracts attribute information (for example, age group, known symptom trends), and reads recent health state information from a cloud database using the user identifier contained in the context. Server performs data aggregation on past symptom records (for example, counting occurrences of “dizziness” over the last three days) and computes summary indicators. Server then concatenates fixed template text with these indicators and attribute information to form a prompt sentence, such as:
“Please generate three short, polite questions in English to check the daily health condition of an elderly person with a recent history of dizziness and reduced appetite. Focus on any change in dizziness, ability to walk, and food intake since yesterday.”

Server outputs this prompt sentence as a text string and a set of generation parameters (for example, maximum length, temperature).

### Step 3:

Server calls the generative AI model to generate question sentences.

Server receives as input the prompt sentence and generation parameters. Server tokenizes the prompt sentence into token IDs and feeds these token IDs into a generative AI model implemented as a transformer-based neural network. Server runs forward propagation through model layers, computing attention weights, intermediate activations, and output token probability distributions at each generation step. Based on these distributions and generation parameters, server selects output tokens (for example, by greedy decoding or sampling), then converts the token IDs back into a text sequence. Server repeats this process to obtain multiple question sentences. Server outputs a list of generated question sentences as plain text strings and sends them to the terminal via the communication network.

### Step 4:

Terminal presents generated question sentences to the user.

Terminal receives as input the list of question sentences from the server. Terminal stores the sentences in a session buffer and selects the first question sentence. Terminal sends the selected text to a display module to render it on the screen and may also send the text to a text-to-speech engine, which converts the characters into phonetic units and synthesized audio waveforms. Terminal outputs visible text and optional audio playback as signals to the display and speaker hardware, thus presenting the question to the user.

### Step 5:

User provides a spoken or typed response.

User receives as input the question sentence via visual and/or auditory presentation. User interprets the question and produces a response by speaking into the microphone or typing on the touch keyboard. User outputs analog voice signals or digital keypress events, which are captured by the terminal's input subsystem.

### Step 6:

Terminal converts the user's response into answer text data.

Terminal receives as input either an audio stream from the microphone or a sequence of characters from the keyboard. When audio is used, terminal digitizes the analog voice signal using an analog-to-digital converter and segments the stream into frames. Terminal computes acoustic features (for example, Mel-frequency cepstral coefficients) and feeds these features to a speech recognition engine. The engine runs a neural network model to map features to phonemes and then to words, generating a text transcript. When text input is used, terminal directly formats the typed characters into a UTF-8 string. Terminal associates the transcript with the current question identifier, user identifier, and timestamp. Terminal outputs an answer text data object containing the question text, the recognized or typed answer text, and metadata.

### Step 7:

Terminal transmits answer text data to the server.

Terminal receives as input the answer text data object. Terminal serializes the object into a structured format such as JSON, including fields for session identifier, question text, answer text, timestamp, and device identifier. Terminal invokes a network client module, opens a secure HTTPS connection to the server endpoint, and sends the serialized data as a request payload. Terminal handles network status and retries if necessary. Terminal outputs the transmitted message onto the communication network and records a local status flag indicating that the answer has been sent.

### Step 8:

Server parses the received answer text data.

Server receives as input the HTTPS request containing the serialized answer text data. Server authenticates the request using tokens or keys in the header, then deserializes the JSON payload into internal data structures, extracting the user identifier, session identifier, question text, answer text, and timestamp. Server validates required fields (for example, non-empty answer text) and logs reception metadata. Server outputs a normalized answer record ready for natural language processing.

### Step 9:

Server applies natural language processing to extract health state information.

Server receives as input the normalized answer record. Server passes the answer text to a natural language processing module that includes a tokenizer, a feature extractor, and one or more classifier or sequence-tagging models. Server converts the text into tokens, maps tokens to embeddings, and feeds the embeddings into a trained neural network classifier that outputs probabilities for multiple symptom categories, emotional states, and severity levels. Server may also run a sequence-tagging model that labels each token with entity tags (for example, “SYMPTOM”, “BODY_PART”, “MODIFIER”). Server combines classifier outputs and entity tags to construct structured health state information, including symptom type, body location, severity score, and an emotional state label, along with confidence values. Server outputs a health state information object linked to the original answer record.

### Step 10:

Server stores structured health state information in a cloud database.

Server receives as input the health state information object and the corresponding answer record. Server opens a database connection to a cloud-based database and constructs write operations, such as insert statements, for tables or collections representing sessions, answers, and symptoms. Server maps the fields in the health state information (for example, symptom type, severity score, timestamp, user identifier) into columns or document fields. Server executes the write operations within a transaction, ensuring consistency between session-level and symptom-level records. Server outputs a confirmation of successful storage and an updated index state that can be used for fast retrieval in subsequent computations.

### Step 11:

Server computes a health risk index from stored records.

Server receives as input a user identifier and a time range or a trigger indicating that new health state information has been stored. Server issues database queries to retrieve multiple health state records and symptom records for the specified user and time window. Server loads these records into memory and constructs feature vectors, such as counts of specific symptoms, average severity scores, trends in symptom frequency, and aggregated emotional state scores. Server feeds these feature vectors into a risk calculation module that may implement a rule engine, a regression model, or a classification model. The module applies weighted sums, thresholds, or model inference to compute a numeric health risk index and an associated risk level category (for example, LOW, MEDIUM, HIGH). Server outputs the current health risk index and risk level for the user.

### Step 12:

Server determines abnormality based on the health risk index.

Server receives as input the health risk index and risk level. Server compares the index against predefined abnormality determination conditions, such as numeric thresholds and trend-based rules (for example, sudden increase in risk index compared to previous sessions).

Server may also check combinations of specific severe symptoms (for example, presence of chest pain and shortness of breath) independent of the aggregate index. Based on these comparisons, server evaluates logical conditions and sets an abnormality flag indicating “normal” or “abnormal/emergency.” Server outputs the abnormality flag and an abnormality summary describing the reason for the determination.

### Step 13:

Server generates a notification message when an abnormality is detected.

Server receives as input the abnormality flag and abnormality summary. When the flag indicates abnormality, server retrieves contact information of family members or healthcare providers from a contact information storage area using the user identifier. Server constructs a notification message by combining a template with dynamic fields, including the user's identifier or alias, latest symptom descriptions, severity levels, and the time of detection.

Server formats the message for one or more channels, such as SMS text, email body, or push notification payload. Server outputs one or more complete notification messages and channel-specific message objects.

### Step 14:

Server transmits the notification message to external destinations.

Server receives as input the channel-specific message objects and contact information. Server selects appropriate communication services, such as an SMS gateway, an email server, or a push notification service. Server establishes connections to these services, formats protocol-specific headers and payloads, and sends the notification messages to the indicated telephone numbers, email addresses, or device identifiers. Server records transmission results and updates the database with notification status, including timestamps and any error codes.

Server outputs confirmation logs and status records that reflect successful or failed delivery.

### Step 15:

Server generates guidance prompt sentences for adaptive feedback.

Server receives as input the health risk index, health state information, and optionally user interaction history. Server constructs a guidance prompt sentence that describes the user's state and requests appropriate feedback text from the generative AI model. For example, server may generate:
“Please generate a short, empathetic advice message in English for an elderly person who reported mild dizziness and leg pain, focusing on safe behavior at home and when to contact a doctor.”

Server encodes relevant structured data (for example, risk level, key symptoms) into the natural-language prompt and may include markers or constraints. Server outputs the guidance prompt sentence and associated generation parameters.

### Step 16:

Server obtains a feedback sentence from the generative AI model.

Server receives as input the guidance prompt sentence and generation parameters. Server tokenizes the prompt, runs it through the generative AI model, and generates a feedback sentence by iteratively sampling output tokens until an end condition is met (for example, maximum length or end-of-sentence token). Server detokenizes the generated tokens into text and performs optional post-processing, such as removing undesired phrases or enforcing length constraints. Server outputs the feedback sentence as plain text.

### Step 17:

Terminal presents the feedback sentence to the user.

Terminal receives as input the feedback sentence from the server. Terminal stores the feedback text in the current session context and passes it to user interface components. Terminal renders the feedback as text on the display and may send it to a text-to-speech engine to synthesize audio. Terminal outputs visual and auditory signals that inform the user of lifestyle precautions or recommendations for contacting a healthcare provider, thereby closing the interaction loop for the current monitoring session.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional health monitoring systems for elderly users primarily rely on static questionnaires, manually configured rule sets, and fixed notification logic. In such systems, a processor typically presents predetermined questions, collects answers, and applies simple threshold comparisons. This architecture has several technical drawbacks when implemented on computing hardware.

First, because the questions are static and not tailored to the evolving condition of each individual, the processor fails to collect information that is optimally informative for subsequent analysis. As a result, the data written to memory and processed by the processor often lacks sufficient granularity or relevance, which reduces the effectiveness and efficiency of later computation, such as time-series analysis or anomaly detection.

Second, existing systems commonly process free-text answers by ad hoc keyword matching or shallow parsing on the server side. These methods are not robust to natural language variation and do not generate structured indicators in a form that is well suited to efficient computation. This causes a technical inefficiency: the processor must repeatedly perform complex, often inaccurate text handling, which increases processing overhead and degrades the quality of the derived health indicators. It also makes it difficult to implement scalable, data-driven algorithms on large, accumulated datasets within the memory system.

Third, notification mechanisms in conventional systems are tightly coupled to fixed thresholds and do not reflect complex combinations or temporal trends of multiple health indicators. As a consequence, the processor either triggers too many false alerts or misses important changes. From a computing perspective, this means that the decision logic executed by the processor does not fully exploit the structured data stored in memory, and thus fails to provide an optimal mapping from analyzed data to control actions such as alert transmission.

Fourth, conventional systems treat question generation and health condition analysis as separate, static modules. The question set is usually not updated based on prior analysis results. Therefore, the processor cannot close the loop between the data collection layer (question presentation) and the data analysis layer (health evaluation). This leads to a technically inefficient feedback loop: the system cannot automatically steer its own input acquisition process to maximize the informativeness of future data, and consequently does not improve its own performance over time.

In addition, many implementations do not provide a standardized pipeline in which a processor: (i) constructs a prompt sentence for a generative artificial intelligence model, (ii) uses the model to dynamically generate question sentences, (iii) normalizes answer data through speech recognition and natural language processing, (iv) stores structured indicators in memory for time-series and statistical analysis, and (v) adapts both notification behavior and subsequent prompt generation based on the detected changes. The lack of such an integrated, feedback-driven architecture results in suboptimal utilization of computing resources, limits scalability, and hinders the ability of the system to accurately and efficiently monitor the health condition of elderly users.

Accordingly, there is a need for an improved computer-implemented system and method in which a processor orchestrates a tight integration between generative artificial intelligence-based question generation, robust natural language analysis, structured health indicator storage, trend-sensitive risk evaluation, and adaptive prompt control. Such an architecture should technically improve how the processor and memory cooperate to collect, transform, store, and analyze health-related data, and how the communication interface is controlled to transmit notifications, thereby improving overall system performance and reliability in monitoring elderly users'health conditions.

The specific processing by the specific processing unit 290 of the data processing device 12 in Example 2 is realized by the following means.

The present invention provides a server comprising a processor and a memory, the processor being configured to (i) construct, based on past health-related information of an elderly user stored in the memory and general health-related knowledge, a prompt sentence that instructs a generative artificial intelligence model to generate at least one question sentence for checking a health condition of the elderly user, and to input the prompt sentence into the generative artificial intelligence model to obtain the at least one question sentence; (ii) control a communication interface to transmit the at least one question sentence to a terminal device via a communication network so that the terminal device presents the at least one question sentence to the elderly user using at least one of a sound output function and a display output function; (iii) receive, from the terminal device, answer data representing at least one of a voice response and a text response from the elderly user, and, when the answer data includes the voice response, execute speech recognition processing to convert the voice response into character information so as to normalize the answer data; (iv) execute a natural language processing engine to analyze the normalized answer data, extract health state indicators including at least one of symptoms, emotions, and time expressions, and generate structured data representing the health state indicators; (v) store the structured data and previously accumulated structured data in the memory and perform at least one of time-series analysis and statistical analysis on the structured data and the previously accumulated structured data to detect a change in the health condition of the elderly user; (vi) determine that the detected change satisfies a predetermined condition corresponding to an abnormal state, and, in response to the determination, control the communication interface to automatically transmit notification information including details of the abnormal state to a related terminal device of a related person; and (vii) update contents of a subsequent prompt sentence to be input into the generative artificial intelligence model, based on the detected change in the health condition and past analysis results stored in the memory, so as to dynamically adapt contents of the at least one question sentence to a current state of the elderly user. This enables an improved computer-implemented health monitoring architecture in which the processor and memory cooperate to form a closed-loop pipeline that (a) dynamically generates context-appropriate question sentences using a generative artificial intelligence model, (b) converts unstructured voice and text responses into structured health indicators via speech recognition and natural language processing, (c) efficiently stores and analyzes such indicators over time using time-series and statistical techniques, (d) executes risk-aware decision logic to control transmission of notifications via the communication interface, and (e) adaptively refines subsequent prompt sentences based on prior analysis, thereby technically enhancing the accuracy, efficiency, and responsiveness of health condition monitoring for elderly users compared to conventional static and non-adaptive systems.

The term “system” refers to a combination of at least one server, at least one terminal device, at least one communication network, and at least one memory device, which cooperate to perform the health monitoring functions described herein.

The term “server” refers to an information processing apparatus, typically including at least one processor, at least one memory, and at least one communication interface, that executes programs to perform data acquisition, analysis, storage, and control related to health monitoring.

The term “processor” refers to a hardware processing unit, such as a central processing unit or other computing circuitry, that executes instructions to perform logical operations, arithmetic operations, data transfer, and control of peripheral components according to programs stored in a memory.

The term “memory” refers to any non-transitory computer-readable storage medium, such as semiconductor memory, magnetic storage, or optical storage, that stores programs, data, prompt sentences, structured data, analysis results, and configuration information for use by the processor.

The term “communication interface” refers to a hardware and software interface, such as a network interface controller or modem and associated protocol stack, that enables the server or terminal device to send and receive data over a communication network.

The term “terminal device” refers to an information processing device operated or carried by an elderly user, such as a mobile device, tablet, or dedicated health monitoring terminal, that is capable of presenting question sentences and acquiring responses.

The term “communication network” refers to any wired or wireless network, including at least one of a local area network, a wide area network, a cellular network, or the Internet, that enables data exchange between the server, terminal devices, and related devices.

The term “elderly user” refers to a human user in an advanced age group who is the subject of health condition monitoring by the system.

The term “generative artificial intelligence model” refers to a machine learning model that, in response to input data such as a prompt sentence, generates new textual content, including question sentences, based on parameters trained on one or more datasets.

The term “prompt sentence” refers to an input text string provided to the generative artificial intelligence model, the text string including at least an instruction and context information that cause the model to generate one or more question sentences appropriate for checking the health condition of the elderly user.

The term “question sentence” refers to a natural-language sentence generated by the generative artificial intelligence model, which is intended to be presented to the elderly user for the purpose of eliciting information about the user's health condition.

The term “past health-related information” refers to data previously acquired and stored regarding the elderly user, including at least one of reported symptoms, historical responses, health indicators, and timestamps associated with such data.

The term “general health-related knowledge” refers to information or patterns relating to health conditions, symptoms, or risk factors that are encoded in the parameters of the generative artificial intelligence model or otherwise available to the processor for constructing prompt sentences.

The term “voice response” refers to an audio signal representing spoken words or utterances by the elderly user in reply to a presented question sentence.

The term “text response” refers to a sequence of characters or symbols input by the elderly user, or derived from speech recognition, as an answer to a presented question sentence.

The term “speech recognition processing” refers to a computational process that converts a voice response into a corresponding text response by analyzing audio signals and identifying words and phrases.

The term “normalized answer data” refers to response data that has been transformed into a standardized text-based format, regardless of whether the original response was a voice response or a text response, so that it can be further analyzed by natural language processing.

The term “natural language processing engine” refers to software and associated models that perform computational analysis of text, including at least one of tokenization, syntactic analysis, semantic analysis, sentiment analysis, and entity extraction, to derive structured information from the text.

The term “health state indicator” refers to a value or label representing an aspect of the elderly user's health condition, including at least one of a symptom type, severity, emotional state, or temporal information, that is derived from the analysis of answer data.

The term “structured data” refers to data formatted in a predefined, machine-readable structure, such as key-value pairs, tables, or records, representing health state indicators in a form suitable for time-series and statistical analysis.

The term “time-series analysis” refers to a computational technique that processes sequences of structured data ordered in time, to identify trends, patterns, or changes in health state indicators across different time points.

The term “statistical analysis” refers to the application of mathematical and statistical methods, such as averaging, variance computation, or threshold-based evaluation, to structured data in order to characterize and detect changes in the elderly user's health condition.

The term “change in the health condition” refers to a variation over time in one or more health state indicators, as detected by time-series analysis or statistical analysis applied to structured data.

The term “abnormal state” refers to a state of the elderly user's health condition in which one or more health state indicators or their change patterns satisfy a predetermined condition indicating increased risk or concern.

The term “notification information” refers to data representing a message that describes at least a detected abnormal state and optionally related context, which is transmitted by the system to a related person or device.

The term “related person” refers to an individual associated with the elderly user, including at least one of a family member, caregiver, or medical professional, who receives notification information from the system.

The term “related terminal device” refers to a terminal device operated by a related person and configured to receive notification information transmitted from the server.

The term “presentation time information” refers to data indicating one or more times at which question sentences are to be presented to the elderly user.

The term “presentation history information” refers to data indicating at least which question sentences were presented, at what times, and optionally whether answers were received, for the elderly user.

The term “presentation schedule” refers to a plan or timetable generated by the processor, based on presentation time information and optionally presentation history information, specifying times at which the terminal device is to present question sentences to the elderly user.

The term “determination logic” refers to a set of computational rules, algorithms, or models executed by the processor to evaluate combinations and trends of health state indicators and to calculate a risk level.

The term “risk level” refers to a quantitative or qualitative measure representing a degree of concern or urgency regarding the elderly user's health condition, as determined by the determination logic based on health state indicators.

In one embodiment, a server, a terminal, and a user cooperate to implement the invention using concrete hardware and software components.

The server includes at least one processor, at least one memory, and at least one communication interface. The server, for example, uses a multi-core central processing unit and an optional graphics processing unit or tensor processing unit for accelerating neural network inference. The memory includes a non-transitory computer-readable storage medium, such as a solid-state drive that stores program modules and data, and a volatile memory such as random access memory used during execution. The server runs an operating system such as a general-purpose server operating system and a web application framework such as a server-side scripting or application platform. The server further uses libraries or services corresponding to a generative AI model and a natural language processing engine.

The terminal includes a processor, a memory, a display, one or more speakers, a microphone, a network interface, and input devices such as a touch panel. The terminal runs a mobile operating system such as a smartphone or tablet system. The terminal installs an application that implements user interaction: presentation of question sentences, voice recording, text input, and secure transmission of answer data to the server.

The user is an elderly person who interacts with the terminal to provide responses. The user hears question sentences through the terminal's speaker or reads them on the terminal's display, and the user responds by speaking into the terminal's microphone or entering text on the terminal's input interface.

The server uses a generative AI model implemented as a neural network, for example a transformer-based sequence-to-sequence model, whose parameters are stored in the server's memory or in a model-serving system accessible via the communication interface. The generative AI model includes multiple encoder and decoder layers, each layer containing multi-head self-attention modules, feedforward networks, layer-normalization units, and learned positional encodings. The model has been trained offline, prior to deployment, using a large corpus of text containing health-related dialogues, symptom descriptions, and general natural language. During training, the server (or an external training system) minimizes a cross-entropy loss function between predicted token sequences and reference question sentences, using gradient-based optimization such as stochastic gradient descent with adaptive learning rate methods. Model parameters such as layer depth, hidden dimension size, attention head count, and vocabulary size are preset, for example 12 or more layers, hidden dimensions of several hundred units, and vocabularies of tens of thousands of subword tokens.

The server constructs a prompt sentence as a text string that concatenates multiple fields: (i) a short system instruction describing the behavior expected from the generative AI model, (ii) a summary of past health-related information of the user, and (iii) optional constraints on language style and output length. The server stores past health-related information in a structured format in the memory, for example in a relational or document-oriented database. Each record may include a user identifier, timestamp, symptom indicators, emotional states, and prior risk levels. The server retrieves a subset of recent records, compresses them into a human-readable summary, and inserts the summary into the prompt sentence.

The server, for example, constructs a prompt sentence such as:
“Act as a caregiver. The user is an elderly person living alone. Recent data: dizziness 3 times in the last 7 days, one headache, poor sleep on 2 days. Generate 3 short, simple daily check questions in English to monitor dizziness, headache, and sleep. Output only the questions.”

The server sends this prompt sentence as input tokens to the generative AI model. The generative AI model, when executed by the processor or by a model-serving engine, performs multiple matrix multiplications and attention score computations across its layers. The model produces output logits for each token position, applies a softmax function to obtain probability distributions, and selects tokens by greedy decoding or sampling. The server receives the generated token sequence, decodes it into text, and splits the text into individual question sentences. These question sentences are stored by the server as records in the memory, associated with user identifiers and timestamps. Because the question sentences are generated conditionally on the summarized health history, they are adapted to the user's current state and thus provide more informative input to subsequent analysis.

The server then uses a communication interface, such as a network adapter and a secure communication protocol, to transmit the generated question sentences to the terminal. The server formats the question sentences into a structured response to an application programming interface request from the terminal. The server may also include metadata such as a preferred presentation time or priority level.

The terminal receives the question sentences over the communication network and stores them locally in a lightweight database. The terminal retrieves configuration settings indicating whether the user prefers voice presentation, text presentation, or both. If voice presentation is enabled, the terminal passes each question sentence to a text-to-speech engine provided by the terminal's operating system. The text-to-speech engine converts the question sentence into an audio waveform through a sequence of digital signal processing operations, such as phoneme mapping, prosody generation, and waveform synthesis using, for example, a parametric or neural vocoder. The terminal plays the synthesized waveform through the speaker, allowing the user to hear the question sentence. If text presentation is enabled, the terminal renders the question sentence as high-contrast text with a large font on the display, improving readability.

The user provides an answer by speaking or typing. When the user speaks, the terminal records the audio via the microphone at a specified sampling rate and bit depth, for example 16 kHz and 16 bits, and encodes the audio into a compressed format such as a standard audio codec. The terminal stores the recorded audio temporarily in the memory and associates it with the question sentence. When the user types a response, the terminal captures the input string in the application and stores it as a text field.

The terminal sends the answer data to the server using an encrypted communication protocol. For voice responses, the terminal may either send the raw audio or, in some embodiments, perform preliminary speech recognition on device using a local speech recognition library. When the audio is sent to the server, the server applies a speech recognition engine that runs acoustic modeling and language modeling to transform the waveform into a text transcription. The speech recognition engine may use a neural acoustic model trained with a connectionist temporal classification loss and a language model trained on large text corpora. The resulting text transcription is merged with any n-best lists or confidence scores and stored as normalized answer data in the memory.

The server then applies a natural language processing engine to the normalized answer data. The natural language processing engine runs on the server's processor and may include one or more of the following components: a tokenization module that segments the text into tokens, a part-of-speech tagging module that assigns syntactic categories, a named-entity recognition module that detects symptom-related expressions, and a sentiment analysis module that computes an emotional polarity score and magnitude. The engine may internally use a neural network architecture such as a bidirectional recurrent network or a transformer-based encoder fine-tuned on annotated datasets of medical and conversational text. The engine outputs a set of health state indicators such as fatigue level, pain presence, symptom onset time, and emotional state. These indicators are represented in a structured data format, for example as key-value pairs: fatigue_score, headache_score, sleep_quality, onset_time, and sentiment_score.

The server stores the structured data in the memory along with a timestamp and user identifier. The server maintains a time-series of structured records, each representing a past interaction. The server executes time-series analysis and statistical analysis on this data: for example, computing moving averages of fatigue scores, counting occurrences of specific symptoms within a sliding window, and computing deviations from baseline values. The server may use algorithms such as exponential moving averages, z-score thresholding, or non-linear scoring functions to detect abnormal patterns. These algorithms operate on compressed, numerical representations of the health state indicators rather than on raw free text, which significantly reduces computational complexity and memory usage.

The server uses determination logic, implemented as a set of rule-based conditions or a trained classifier, to evaluate combinations of indicators and their trends. For example, the server may define rules such as:
if fatigue_score≥a threshold and headache_score≥a threshold for multiple consecutive days, and sentiment_score is negative, then classify the state as high risk;
if dizziness-related indicators increase over several interactions while sleep_quality deteriorates, then classify the state as moderate risk.

The server executes these rules on the structured data stored in memory, calculating an overall risk level. The risk level is stored and used to control notification behavior.

When a risk level satisfies a condition corresponding to an abnormal state, the server prepares notification information that includes the type of abnormality, the relevant indicators, and timestamps. The server uses its communication interface to send this notification to a related terminal, such as a device used by a family member or caregiver. The server may send the notification via text message, email, or push notification services. Because the server operates on structured indicators and precomputed trends, the notification decision can be made with reduced latency and reduced false positives compared to simple threshold checks, improving both processing speed and accuracy.

The server also updates subsequent prompt sentences based on analysis results. The server aggregates recent structured data and determines which indicators are currently most unstable or most critical. The server then constructs a new prompt sentence that emphasizes these indicators. For example, after detecting increasing fatigue and recurring headaches, the server may construct a prompt sentence such as:
“Act as a caregiver. The user is an elderly person whose recent data shows increasing fatigue over 3 days and recurring mild headaches. Generate 3 short, simple daily check questions in English that focus on fatigue level, headache intensity, and any new symptoms. Output only the questions.”

The generative AI model, when conditioned on this prompt sentence, generates question sentences that specifically probe the user's fatigue, headache, and any new complaints. This feedback loop allows the server to adapt the question content dynamically, thereby optimizing the information collected per interaction.

The described architecture improves computer technology in several ways. The server reduces communication load by transmitting compact, structured indicators and trend summaries instead of raw transcripts and audio for downstream processing. The server reduces processing overhead on the natural language analysis by normalizing responses once and storing structured representations, allowing later computations to operate on lightweight numerical data. The adaptive prompt generation reduces redundant or uninformative questions, thereby decreasing the number of interactions required to detect an abnormal state and improving response time. The use of a transformer-based generative AI model and a dedicated natural language processing engine, applied in a specific pipeline with structured storage and trend-based risk calculation, yields higher detection accuracy and better use of memory resources compared to naive keyword or rule-based systems.

The server and terminal perform specific data processing steps that are not merely automating human judgment. The server applies machine-learned models that exploit high-dimensional parameter spaces and attention mechanisms to derive context-sensitive question sentences that humans cannot easily maintain in a consistent, data-driven manner over long-term histories. The server maps unstructured voice and text into structured features using neural sequence models, then applies quantitative time-series algorithms and rule-based logic to control physical outputs, namely notifications delivered through communication hardware and question presentations rendered on terminal hardware. This combination changes the manner in which the computer itself stores, retrieves, and processes health-related data, leading to improved computational efficiency and technical reliability.

In an alternative embodiment, the generative AI model may be hosted on an external model-serving platform, and the server may send prompt sentences and receive generated question sentences via an application programming interface. In another embodiment, the natural language processing engine may be integrated with the generative AI model such that a single neural architecture is fine-tuned for both question generation and answer analysis, with shared embeddings and layers but separate task-specific output heads. In another embodiment, the terminal may perform on-device speech recognition and basic natural language parsing, thus reducing bandwidth usage by sending preprocessed structured data to the server.

In yet another embodiment, the determination logic may be realized as a machine-learned classifier, such as a gradient boosted decision tree or a shallow neural network, trained on historical structured records to predict a risk level. The server may periodically retrain the classifier using accumulated data, storing updated model parameters in the memory and applying them to future interactions. This retraining process may be scheduled or may be triggered when a sufficient volume of new data is available, thereby improving detection performance over time without changing the external behavior of the system.

Through these embodiments, the server, terminal, and user cooperate in a concrete architecture in which generative AI-based prompt sentence construction, structured health indicator extraction, time-series analysis, and adaptive notification control are implemented as specific data flows through hardware and software modules. This yields technical effects including improved detection accuracy, reduced computation time for analysis, more efficient data storage and retrieval, and reduced communication load, thereby constituting more than an abstract idea or simple automation of human activity.

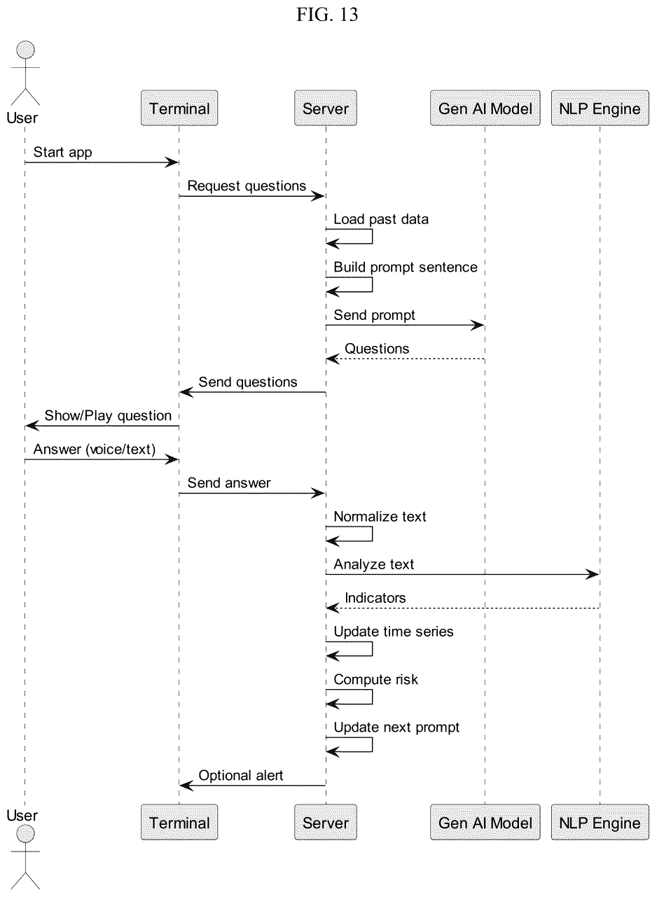

The following describes the processing flow using FIG. 13.

### Step 1:

Server retrieves and summarizes past health-related data.

Server uses a database interface to read past records for a target user from a storage system.

Input: user identifier and a time range (for example, last 7 or 30 days).

Server executes a database query to obtain fields such as timestamps, symptom indicators (fatigue, headache, dizziness), sleep quality scores, and previous risk levels.

Server performs data processing by aggregating these records: calculating counts (for example, “dizziness 3 times”), averages (for example, “sleep quality score 2.3”), and detecting recurring symptoms.

Output: a compact health summary string, for example, “dizziness 3 times in the last 7 days, one headache, poor sleep on 2 days,” and a structured summary object stored in memory.

### Step 2:

Server constructs a prompt sentence for the generative AI model.

Input: health summary string from Step 1, static instruction templates, and user profile information (age, living alone status).

Server concatenates these elements into a single text sequence, inserting markers or delimiters as needed.

Server performs string processing operations such as template filling (replacing placeholders with actual values) and length checks.

Output: a prompt sentence, for example, “Act as a caregiver. The user is an elderly person living alone. Recent data: dizziness 3 times in the last 7 days, one headache, poor sleep on 2 days. Generate 3 short, simple daily check questions in English to monitor dizziness, headache, and sleep. Output only the questions.”

### Step 3:

Server sends the prompt sentence to the generative AI model and receives generated question sentences.

Input: prompt sentence from Step 2.

Server tokenizes the prompt sentence into token IDs using the generative AI model's vocabulary, then calls a model inference routine (for example, a transformer-based encoder-decoder network).

Server performs numerical data operations including matrix multiplications, attention score calculations, and softmax operations across multiple layers to compute output token probabilities.

Server decodes the resulting token sequences into text and splits the generated text into separate lines or question boundaries.

Output: a list of question sentences, for example,
“Have you felt dizzy today?”
“Have you had any headaches in the last 24 hours?”
“Did you sleep well last night?”

### Step 4:

Server validates and stores the generated question sentences.

Input: raw list of question sentences from Step 3.

Server checks each question sentence for length limits, forbidden terms, language consistency, and duplication.

Server performs data filtering and cleaning by discarding or modifying sentences that fail validation and re-indexing the remaining ones.

Server writes validated question sentences into the database, associating each with a user identifier, creation timestamp, and internal question ID.

Output: a validated and stored question set, and a response payload ready to be sent to the terminal.

### Step 5:

Terminal requests and receives question sentences from the server.

Input: API request containing user identifier and current date/time parameters.

Terminal sends a request through a communication interface to the server, then receives a response that includes question IDs and texts.

Terminal parses the response format, mapping each question ID to its corresponding text and any metadata such as recommended time of day.

Terminal stores the question set in local storage (for example, a small database or key-value store).

Output: a local question list on the terminal, ready for scheduling and presentation.

### Step 6:

Terminal schedules presentation of the question sentences.

Input: local question list from Step 5 and presentation time preferences (for example, morning, afternoon, evening) stored in terminal settings.

Terminal computes specific timestamps for each question, possibly spreading them over the day.

Terminal registers alarms or notifications with the operating system's scheduler, each alarm linked to a question ID.

Output: a schedule of alarms in the terminal's OS, each capable of triggering presentation of the corresponding question sentence at the proper time.

### Step 7:

Terminal presents a question sentence to the user.

Input: triggered alarm event containing a question ID.

Terminal retrieves the matching question sentence from local storage and checks the current presentation mode (voice, text, or both).

If voice mode is active, terminal calls a text-to-speech engine, which converts the question text into an audio waveform; terminal then routes the waveform to the speaker hardware.

If text mode is active, terminal updates a display widget with the question text, adjusting font size and contrast according to accessibility settings.

Output: a presented question, either spoken through the speaker or shown on the display, available for the user to perceive.

### Step 8:

User provides an answer via voice or text input.

Input: presented question from Step 7.

User listens to the spoken question or reads the text, then decides on an answer such as “I had a slight headache this morning but feel better now.”

If speaking, user presses a record or answer button and talks into the microphone until finished, then stops recording.

If typing, user taps a text input area and enters an answer string using the terminal's on-screen keyboard, then submits the answer.

Output: a raw user response in either audio form (recorded waveform) or text form (input string) held by the terminal.

### Step 9:

Terminal records, normalizes, and packages the user's answer.

Input: raw audio data or raw text data from Step 8.

For audio input, terminal encodes the waveform into a compressed audio file format and attaches metadata such as user ID, question ID, and timestamp.

For text input, terminal constructs a response object containing the text string and the same metadata.

Terminal may optionally perform on-device speech recognition on the audio to produce preliminary text, in which case it aligns recognized text with the original audio.

Terminal packs the answer data and metadata into a structured message (for example, an HTTP body) ready for transmission.

Output: a normalized response package that includes either audio plus metadata or text plus metadata.

### Step 10:

Terminal sends the response package to the server.

Input: normalized response package from Step 9 and network status information.

Terminal checks network connectivity; if available, terminal opens a secure connection to the server's endpoint and transmits the package.

Terminal waits for an acknowledgment from the server; on success, terminal updates local status to mark the response as delivered, or queues it for retry when transmission fails.

Output: a response record received at the server side, ready for analysis, and an updated delivery state on the terminal.

### Step 11:

Server receives and converts the user's answer into normalized text.

Input: response package from Step 10, containing audio or text and metadata.

If the response includes audio, server passes the audio to a speech recognition engine, which computes acoustic features, aligns them with phonetic models, and decodes them into a text transcription.

If the response includes text, server validates the text and normalizes character encoding and punctuation.

Server associates the normalized text with user ID, question ID, and timestamp, and stores this in memory as normalized answer data.

Output: normalized text answer linked with metadata, ready for natural language analysis.

### Step 12:

Server analyzes the normalized text using a natural language processing engine.

Input: normalized text answer and, optionally, context data such as the original question sentence.

Server tokenizes the text into words or subword units, assigns part-of-speech tags, and runs entity recognition to detect symptom-related phrases (for example, “headache,” “tired,” “this morning”).

Server uses sentiment or emotion analysis to calculate a sentiment score and magnitude representing the user's subjective feeling.

Server maps the detected entities and sentiment values to numeric health state indicators such as fatigue_score, pain_intensity, and onset_time.

Output: a structured set of health state indicators with associated values and an analysis log kept in memory.

### Step 13:

Server stores structured indicators and updates a time-series history.

Input: structured health state indicators from Step 12 and the user's existing records.

Server inserts a new record into a time-series data structure that includes timestamp, symptom scores, emotion scores, and risk flags.

Server retrieves recent records for the same user and computes moving averages, trend slopes, and deviations from baseline values using arithmetic operations.

Server updates aggregate statistics in the database (for example, number of days with headache in the last 14 days) to support fast retrieval.

Output: an updated longitudinal dataset that reflects the user's latest health state and computed trends.

### Step 14:

Server evaluates risk using determination logic based on indicators and trends.

Input: current indicators and trend metrics from Step 13.

Server applies rule-based conditions or classifier outputs, combining multiple indicators and their changes over time to compute a risk level.

Server may, for example, calculate a composite score as a weighted sum of fatigue_score, pain_intensity, and negative sentiment, then compare this score to pre-defined thresholds.

Server classifies the situation into categories such as “normal,” “watch,” or “abnormal,” and stores the risk level in memory.

Output: a risk assessment result, including a discrete risk level and underlying reasons (which indicators triggered the decision).

### Step 15:

Server generates and sends notification information when an abnormal state is detected.

Input: risk assessment result from Step 14 and contact information for related persons.

If the risk level meets or exceeds an abnormal threshold, server builds a notification message that describes the abnormal pattern, for example, “The user has reported increasing fatigue and a headache this morning, a significant change from recent days.”

Server uses its communication interface to send the notification to a related terminal via messaging or push services, encoding the message into the required format and addressing it to the appropriate destination.

Output: transmitted notification information received by the related terminal, and a log of the notification stored on the server.

### Step 16:

Server updates future prompt sentence generation based on analysis results.

Input: structured indicators, risk history, and question logs stored in memory.

Server identifies which health state indicators show unstable or worsening trends and counts how often particular symptoms appear in recent answers.

Server recomputes or adjusts prompt sentence templates to emphasize those indicators; for example, if dizziness and poor sleep become more frequent, server inserts these topics into the next prompt sentence.

Server constructs a new prompt sentence such as, “Act as a caregiver. The user is an elderly person whose recent data shows increasing fatigue over 3 days and recurring mild headaches. Generate 3 short, simple daily check questions in English that focus on fatigue level, headache intensity, and any new symptoms. Output only the questions.”

Output: an updated prompt sentence that will be used in a subsequent execution of Step 3 to generate new, more targeted question sentences.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional computer-implemented health monitoring systems for elderly persons typically collect simple questionnaire data and store it as static records. In many cases, such systems rely on fixed question sets, shallow keyword-based analysis, and manual rule configuration. As a result, these systems suffer from several technical problems in terms of information processing performance and adaptability.

First, because conventional systems use static questions that are not adapted to an individual user's historical data or emotional context, the input data obtained from the user is often sparse, redundant, or poorly targeted. This degrades the information density of collected data and leads to inefficient use of computational and communication resources, since the system repeatedly processes low-value data while failing to capture subtle but important changes in the user's state.

Second, prior systems often treat speech, text, and emotional cues as separate data streams or ignore some of them entirely. Audio data may be transcribed into text, but the combined contextual meaning, including emotional nuances present in voice or facial expressions, is not effectively integrated into the analysis pipeline. This fragmented or incomplete processing causes the analysis unit to misclassify or overlook early anomalies, which in turn forces repeated polling, redundant transmissions, and increased server load to compensate for low detection accuracy.

Third, many existing architectures handle anomaly detection and notification as a simple thresholding mechanism on isolated measurements. These architectures do not employ a unified representation that fuses historical state information, current natural language responses, and emotion-derived features. As a result, anomaly detection logic is either over-sensitive or under-sensitive, leading to unnecessary alerts or missed events. This behavior reduces the practical reliability of automated notification mechanisms and imposes additional processing and communication overhead when alerts must later be filtered or manually corrected.

Fourth, conventional question-generation mechanisms, if present at all, are not implemented as feedback-driven generative models that leverage prior interaction data. Question content is usually pre-authored or heuristically selected and is not dynamically tuned based on longitudinal history and current emotional state. This lack of adaptive question generation prevents the system from steering the user dialogue toward the most diagnostically informative topics, thereby limiting the efficiency of the data acquisition pipeline and wasting processing cycles on routine or irrelevant exchanges.

In summary, there is a need for an improved computer-implemented system that: (i) uses a generative model to dynamically generate and adapt question sentences based on historical state and emotional context, (ii) integrates multimodal information including speech, text, and emotion features into a unified analysis and anomaly detection framework, and (iii) automatically controls notification content and routing based on fused analysis results, in order to improve the quality, efficiency, and reliability of health and emotion monitoring for elderly users from a computing-architecture standpoint.

The specific processing by the specific processing unit 290 of the data processing device 12 in Application Example 2 is realized by the following means.

The present invention provides a server comprising a processor, a memory, at least one communication interface configured to communicate with at least one terminal device, and at least one program stored in the memory and executable by the processor, wherein the processor is configured to execute instructions that cause a generative information processing model to generate, in response to a prompt sentence including instruction content for creating inquiry information for confirming physical and psychological states of an elderly user, at least one question sentence to be presented to the elderly user, to control the terminal device to present the generated question sentence as audio information and/or character information and to collect response information including audio information and/or character information from the elderly user, to convert, by using an acoustic recognition processing unit, the audio information included in the response information into text information, to analyze, by using an analysis processing unit having a natural language processing function and the generative information processing model, the text information to extract state information relating to the physical state and the psychological state of the elderly user and to generate analysis result information including risk information and abnormality information, to store, in the memory acting as a storage management unit, the state information, the risk information, and the abnormality information as history information and to evaluate a temporal change in the state of the elderly user based on comparison between current history information and past history information, to estimate, by using an emotion estimation processing unit and feature information extracted from the audio information and/or image information, an emotional state of the elderly user and to associate and record the emotional state with the state information in the memory, and to execute abnormality determination processing based on at least the analysis result information, the emotional state, and the history information and, when an abnormal condition is determined to be present, to generate notification information including alarm information and to transmit the notification information automatically via the communication interface to an information processing terminal of a related person of the elderly user. This enables a computer system to dynamically tailor question generation based on fused historical and emotional context, to integrate multimodal response data into a unified analysis pipeline for more accurate anomaly detection, and to automatically control notification content and routing, thereby improving overall efficiency, responsiveness, and reliability of health and emotion monitoring operations executed by the server.

The term “processor” refers to a hardware computation element, such as a central processing unit or other programmable processing circuitry, that executes instructions to perform data processing, control operations, and communication control in the system.

The term “memory” refers to one or more hardware storage elements, such as volatile or non-volatile semiconductor storage devices, configured to store programs, parameters, models, and data used and generated by the processor.

The term “terminal device” refers to a user-side electronic apparatus having at least a communication function and an input/output interface, such as a portable communication device or a display-equipped communication device, that presents information to an elderly user and acquires response information from the elderly user.

The term “communication interface” refers to a hardware and/or software communication unit that enables data transmission and reception between the server and external devices or networks via wired or wireless communication protocols.

The term “generative information processing model” refers to a machine learning model or other computational model that receives an input text including a prompt sentence and generates output text such as question sentences or analysis results based on learned statistical or rule-based relationships.

The term “prompt sentence” refers to an input text string including instruction content that specifies a task or constraint for the generative information processing model, such as generating health-related questions or structuring analysis results.

The term “question sentence” refers to a text string generated by the generative information processing model that is intended to be presented to an elderly user for inquiring about the user's physical state and psychological state.

The term “audio information” refers to digital data representing sound, including spoken utterances of the elderly user and synthesized or recorded speech presented to the elderly user.

The term “character information” refers to digital data representing textual content encoded as machine-readable characters for display or further processing.

The term “response information” refers to information obtained from the elderly user in response to a question sentence, including at least audio information and/or character information that expresses the user's answer.

The term “acoustic recognition processing unit” refers to a functional component, implemented by software executed by the processor and/or dedicated hardware, that analyzes audio information and converts spoken content into corresponding text information.

The term “text information” refers to character-based data produced by the acoustic recognition processing unit or other text-generation mechanisms that represent the linguistic content of a spoken or written response.

The term “analysis processing unit” refers to a functional component, implemented by software executed by the processor and/or dedicated hardware, that performs natural language processing on text information to extract structured state information and to generate analysis result information.

The term “natural language processing function” refers to a computational capability for analyzing human language text, including at least tokenization, parsing, semantic interpretation, and information extraction for health-related and emotion-related content.

The term “state information” refers to structured data elements describing aspects of an elderly user's physical state and psychological state derived from analysis of the user's response information.

The term “physical state” refers to bodily conditions of the elderly user, including symptoms, discomforts, or other health-related characteristics expressed in the user's responses.

The term “psychological state” refers to mental or emotional conditions of the elderly user, including feelings, mood, stress, or anxiety expressed or inferred from the user's responses.

The term “analysis result information” refers to structured output data generated by the analysis processing unit and/or the generative information processing model, including at least state information, risk information, and abnormality information.

The term “risk information” refers to data indicating a degree or level of health-related or emotion-related risk for the elderly user as evaluated from the state information and other analysis.

The term “abnormality information” refers to data indicating presence or absence of an abnormal condition in the elderly user's physical state or psychological state as determined by analysis.

The term “history information” refers to accumulated records stored in the memory that include past state information, risk information, and abnormality information associated with corresponding points in time.

The term “storage management unit” refers to a functional component, implemented by the memory and control logic of the processor, that manages writing, reading, and organizing of history information and other data.

The term “temporal change” refers to variation over time in one or more elements of an elderly user's state information, risk information, abnormality information, or emotional state derived from multiple instances of history information.

The term “emotion estimation processing unit” refers to a functional component, implemented by software executed by the processor and/or dedicated hardware, that estimates an emotional state of the elderly user based on feature information extracted from audio information and/or image information.

The term “audio feature information” refers to parameterized representations of audio information, including at least pitch, energy, temporal dynamics, and spectral characteristics used for emotion estimation.

The term “image feature information” refers to parameterized representations of image information, including at least facial landmarks, expressions, or motion characteristics used for emotion estimation.

The term “image information” refers to digital data representing visual content, such as images or video frames, acquired from a camera of the terminal device.

The term “emotional state” refers to a classification or vector of values representing inferred emotions of the elderly user, such as calmness, anxiety, sadness, or stress, obtained by the emotion estimation processing unit.

The term “abnormality determination processing” refers to a sequence of computational operations that evaluates analysis result information, emotional state, and history information to determine whether an abnormal condition is present.

The term “notification information” refers to structured data generated by the processor that includes at least alarm information and descriptive content intended to be transmitted to a related person of the elderly user.

The term “alarm information” refers to data indicating that an abnormal condition or elevated risk related to the elderly user has been detected and that attention or intervention may be required.

The term “communication processing unit” refers to a functional component associated with the communication interface that controls formatting, transmission, and reception of messages, including notification information, over one or more communication networks.

The term “information processing terminal” refers to an external computing device associated with a related person of the elderly user, such as a general-purpose computing device or communication device configured to receive and present notification information.

The term “related person” refers to a person who is associated with the elderly user, such as a family member, caregiver, or medical staff member, and is designated as a recipient of notification information.

In one embodiment, a server, a terminal, and a user cooperate to realize the claimed system.

### A. Overall Hardware and Software Configuration

The server comprises at least one processor, at least one memory, at least one non-transitory storage, and at least one communication interface. The server executes an operating system such as a general-purpose server operating system and runs multiple software modules, including a web server module, an application server module, a speech recognition client module, a natural language processing module, a generative AI model client module, an emotion estimation module, a data management module, and a notification control module.

The terminal comprises at least one processor, at least one memory, an audio input device such as a microphone, an audio output device such as a speaker, a display device, an optional image capture device such as a camera, and a wireless or wired communication interface. The terminal executes a mobile operating system and runs an application program that communicates with the server, presents question sentences to the user, acquires response information, and transmits the response information to the server.

The user is an elderly person who interacts with the terminal by listening to audio output, reading text on the display, and providing spoken or textual responses regarding physical and psychological states.

### B. Generative AI Model and Prompt Sentence Configuration

The server uses a generative AI model as a generative information processing model. In one embodiment, the generative AI model is implemented as a transformer-based neural network comprising an encoder-decoder or decoder-only architecture with multiple attention layers, feedforward layers, and normalization layers. The model parameters comprise a set of weight matrices and bias vectors stored in the memory and loaded into the processor at runtime.

The server configures the generative AI model with a vocabulary and tokenization scheme, such as subword units. The server employs a prompt sentence as input to the generative AI model to control generation behavior. The prompt sentence includes instruction content that specifies a task, such as generating question sentences for confirming an elderly user's health state or structuring analysis results.

In one example, the server uses a prompt sentence:
“Generate five short, polite questions to check the daily physical and emotional health of an elderly person living alone.”

In another example, the server uses a prompt sentence:
“You are a health monitoring assistant. Read the elderly person's statement and extract: (1) physical symptoms, (2) emotional state, (3) overall risk (low/medium/high), (4) whether to notify family (yes/no) with a short reason. Output the items in labeled lines.”

The server provides the prompt sentence as text to the generative AI model, and the generative AI model outputs text sequences that are decoded into question sentences or structured analysis result information.

The server trains or fine-tunes the generative AI model on domain-specific corpora that include dialog data about health status, descriptions of symptoms, emotional expressions, and expert annotations of risk levels. The server uses a loss function such as cross-entropy between predicted token distributions and ground-truth tokens. The server updates model weights using gradient-based optimization, such as stochastic gradient descent or adaptive moment estimation. The server may use data augmentation techniques such as paraphrasing or synonym replacement to increase robustness of the generative AI model to variations in user language.

By structuring the generative AI model in this way, the server improves accuracy and stability of generated question sentences and analysis results compared to rule-based or template-based generation.

### C. Question Generation and Adaptation Based on History and Emotion

The server stores, in the memory, history information for each user. The history information includes past state information records, risk information, abnormality information, and emotional state labels with timestamps. The server organizes this history information in a structured data store, such as a relational table or a document collection, with indexed keys for user identifier, date, and symptom categories.

The server uses the history information and the current emotional state of the user to construct a context-enhanced prompt sentence for the generative AI model. For example, when the user has frequently reported headaches and elevated anxiety in the past week, the server includes a summary text in the prompt sentence:
“Past 7-day summary: headache reported on 4 days, sleep problems on 3 days, emotional state often anxious. Generate three short follow-up questions focusing on headaches, sleep quality, and stress management for an elderly person.”

The server builds this summary using a deterministic aggregation algorithm that counts symptom occurrences and selects the top-k frequent symptoms. The server also maps emotional state scores to descriptive phrases. The server then concatenates the summary and the instruction text into a single prompt sentence.

By altering the prompt sentence in this non-conventional, feedback-driven way, the server causes the generative AI model to output question sentences that are targeted to the user's current and past health profile. This improves the information density of user responses, reduces redundant questioning, and reduces bandwidth and processing load by avoiding unnecessary or irrelevant questions.

### D. Acquisition and Processing of Multimodal Response Information

The terminal presents generated question sentences as both character information and audio information. The terminal renders the character information on the display and uses text-to-speech software to synthesize audio information from the question sentence text. The terminal outputs the audio information through the speaker so that the user can listen.

The user speaks an answer, such as “I have a slight headache and I feel a little anxious today,” into the microphone. The terminal acquires the spoken response as raw audio data.

The terminal may also acquire image information from an integrated camera, capturing the user's face while speaking.

The terminal encapsulates the audio information and optional image information as response information, adds metadata such as timestamp and user identifier, and transmits the response information to the server using a communication protocol such as an encrypted network protocol. This transmission uses the communication interface of the terminal and the communication interface of the server.

The server receives the response information and stores the raw audio and image data in storage. The server then invokes an acoustic recognition processing unit implemented as a client for a speech recognition engine. The acoustic recognition processing unit extracts acoustic feature sequences from the audio information, such as mel-frequency cepstral coefficients, energy, and temporal derivatives, and feeds these features into an acoustic model such as a deep neural network trained for speech recognition. The acoustic model outputs phonetic or character probabilities, and a decoding algorithm generates text information representing the recognized utterance of the user.

The server uses an emotion estimation processing unit to process acoustic and image feature information. The emotion estimation processing unit extracts prosodic features such as pitch contour, speaking rate, and intensity from the audio information and facial action unit features from the image information using a convolutional or recurrent neural network. The emotion estimation processing unit classifies the emotional state of the user into one or more labels such as calm, anxious, sad, or stressed, with corresponding confidence scores.

By combining both lexical content (text information) and paralinguistic signals (audio and image features), the server obtains a richer representation of the user's condition than is possible with purely text-based analysis. This multimodal fusion improves the accuracy of state estimation and anomaly detection, which directly reduces false alarms and missed events, thereby improving system reliability.

### E. Natural Language Analysis and Anomaly Determination

The server uses an analysis processing unit having a natural language processing function to analyze the text information. The analysis processing unit may include a tokenizer to segment text into tokens, a part-of-speech tagger, a syntactic parser, and a semantic analyzer that identifies entities, symptoms, and negations. The server may store domain dictionaries of symptom expressions and severity modifiers to improve extraction performance.

The server feeds the text information, possibly combined with short summaries of history information, into the generative AI model with a prompt sentence such as:
“You are a health monitoring assistant. Read the elderly person's statement and extract: (1) physical symptoms, (2) emotional state, (3) overall risk (low/medium/high), (4) whether to notify family (yes/no) with a short reason.”

The generative AI model outputs an analysis result in which symptoms, emotional descriptors, risk level, and notification recommendation are explicitly indicated. The server parses this output, for example by detecting labeled lines or markers, and converts the textual output into structured data fields for state information, risk information, and abnormality-related information.

The server stores these fields as history information together with the emotional state determined by the emotion estimation processing unit. The server maintains per-user time series of symptoms and emotional states. The server applies anomaly determination processing, which may include rule-based criteria, statistical deviation detection, and thresholding on risk scores. For instance, the server may determine that a chronic issue is present when a particular symptom appears on more than a configured number of days within a rolling window and when the emotional state is consistently negative.

This abnormality determination processing reduces noise by aggregating evidence over time and using both content and emotion signals, instead of relying on isolated single-session observations. This yields more accurate anomaly detection and allows the server to issue fewer but more meaningful notifications.

### F. Notification Control and Communication Efficiency

When the server determines that an abnormal condition is present, the server generates notification information containing alarm information and explanatory text about the detected condition. The server uses the communication processing unit to send this notification information to an information processing terminal of a related person, such as a caregiver or family member. The server selects one or more destinations and communication channels based on configuration and severity level, such as short-range wireless communication within a local network, mobile communication, or a data network.

Because the server uses structured risk information and detailed history information, the server can include concise yet informative summaries in notification messages. This reduces the need to transmit raw data or verbose reports, thereby reducing communication load and improving responsiveness.

### G. Technical Improvements Over Conventional Systems

The server improves computer technology in several ways. First, dynamic generation of question sentences using the generative AI model and prompt sentences that incorporate history information and emotional state improves data acquisition efficiency. The server steering mechanism reduces redundant or uninformative interactions, thereby decreasing storage and network usage while maintaining or increasing diagnostic accuracy.

Second, integration of multimodal features (speech content, acoustic features, and image-based facial features) in a unified analysis pipeline improves classification accuracy of physical and emotional states compared to systems that only use text or predefined questionnaires. This increased accuracy allows the server to use simpler downstream rule sets and to avoid heavy manual tuning, thus reducing computational cost and maintenance overhead over the lifetime of the system.

Third, the server's internal data structures and algorithms provide technical benefits. The server maintains histories with indexed symptom counts, time-stamped emotional state vectors, and derived aggregates, and then uses these structures to condition prompt sentences and anomaly rules. This design reduces query time for historical pattern detection and enables real-time or near-real-time anomaly detection even on resource-constrained hardware.

Fourth, the generative AI model is not used merely as a black-box replacement of human reasoning. Instead, the server constrains and orchestrates the generative AI model by providing structured prompt sentences, by enforcing output formats that can be parsed deterministically, and by combining model outputs with hand-crafted anomaly rules and multimodal features. This hybrid architecture enables the server to perform non-intuitive, non-conventional processing that a simple human-mimicking automation would not perform, such as dynamically re-weighting question topics based on aggregated feature statistics and model uncertainty.

Fifth, the server's training and adaptation processes result in improvements over time. The server can log model outputs and subsequent ground-truth corrections, and can use these logs to fine-tune the model with an updated loss function that penalizes misclassification of high-risk states more heavily. This re-weighted loss improves safety-critical performance and yields quantifiable reductions in error rates for severe conditions.

### H. Alternative Embodiments and Variations

The server can employ different generative AI model architectures. In another embodiment, the server uses a sequence-to-sequence encoder-decoder model with attention, where the encoder ingests a concatenation of the prompt sentence and a serialized history representation, and the decoder outputs question sentences or analysis results. In yet another embodiment, the server uses separate models: a first generative model specialized for question generation, and a second model specialized for analysis, both driven by tailored prompt sentences.

The terminal can be implemented as a stationary home device, a wearable device, or an integrated device in a living environment, as long as it can present question sentences and capture response information with sufficient quality. The terminal may pre-process audio or image data, such as performing noise reduction or compression, before transmission to the server to further reduce bandwidth usage and to improve recognition accuracy.

The user can interact in multiple languages, and the server can maintain different generative AI model instances or multilingual embeddings to support cross-lingual operation. The server may also adapt prompt sentences dynamically according to the user's language preference and comprehension level, such as generating shorter or simpler question sentences for users with limited cognitive capacity.

By implementing these embodiments, the server, the terminal, and the user form a coordinated system that not only automates data collection but also improves core computer functions, including data processing efficiency, communication optimization, storage management, and pattern recognition accuracy, thereby providing a technical solution that goes beyond mere automation of a human health interview.

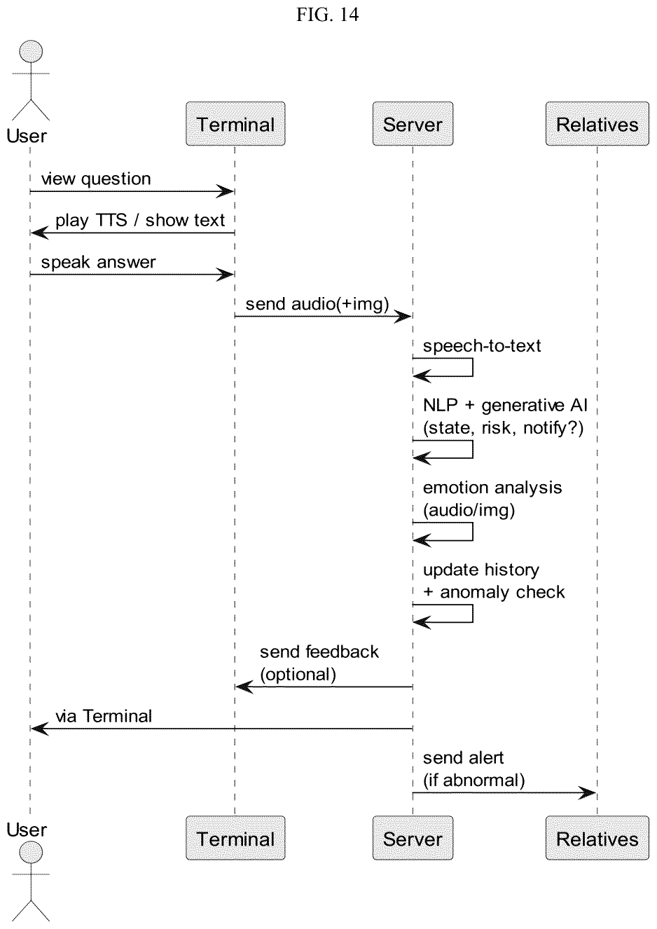

The following describes the processing flow using FIG. 14.

### Step 1:

Server prepares historical context and emotional profile.

Server receives, as input, history information stored in memory for a given user, including past state information, risk information, abnormality information, and previously estimated emotional states. Server reads these records from a structured data store using the user identifier. Server performs data aggregation operations, such as counting occurrences of each symptom in a recent time window (for example, last 7 days), computing average risk level, and summarizing dominant emotional labels. Server outputs a compact context summary string and a structured context object containing symptom counts and emotion statistics.

### Step 2:

Server constructs a prompt sentence for a generative AI model.

Server receives, as input, the context summary and a stored instruction template for question generation. Server concatenates the context summary with the instruction template to build a prompt sentence, for example: “Past 7-day summary: headache reported on 4 days. Generate three short, polite questions focusing on headaches and sleep quality for an elderly person living alone.” Server performs string formatting operations and inserts recent symptom names and counts into the prompt sentence. Server outputs a finalized prompt sentence as text.

### Step 3:

Server generates targeted question sentences using a generative AI model.

Server receives, as input, the prompt sentence and sends it to the generative AI model. Inside the model, the prompt sentence is tokenized into subword tokens, mapped to embeddings, and processed through multiple transformer layers that perform attention and feedforward computations to predict next-token probability distributions. Server decodes the resulting token sequences into natural language question sentences. Server outputs a set of question sentences, each as a text string, and stores them temporarily in memory.

### Step 4:

Server transmits question sentences to the terminal.

Server receives, as input, the generated question sentences and the terminal identifier associated with the user. Server wraps the questions into a message payload that includes a session identifier and metadata such as language and display options. Server uses the communication interface to send the payload to the terminal via a network protocol. Server outputs a successfully sent message status or an error status to its logging subsystem.

### Step 5:

Terminal presents the question sentences to the user.

Terminal receives, as input, the message payload containing question sentences and the session identifier. Terminal parses the payload and selects the current question sentence to present. Terminal converts the text into audio using a text-to-speech engine, generating a waveform from phonetic and prosodic parameters. Terminal outputs both character information on the display and audio information from the speaker so that the user can read and hear the question.

### Step 6:

User provides a spoken response.

User receives, as input, the question sentence via the terminal's audio and display. User mentally interprets the question and produces a spoken response, such as “I have had a slight headache for three days and I feel anxious.” User outputs the spoken utterance as acoustic sound directed toward the terminal's microphone.

### Step 7:

Terminal captures audio (and optionally image) response information.

Terminal receives, as input, the user's spoken sound signal via the microphone and optionally the user's facial image via the camera. Terminal samples the sound into digital audio frames and compresses them using an audio codec if configured. Terminal may capture image frames at a fixed frame rate and encode them using an image or video codec. Terminal packages the audio data, optional image data, session identifier, and timestamps into response information. Terminal outputs the response information as a data packet ready for transmission.

### Step 8:

Terminal transmits response information to the server.

Terminal receives, as input, the response information packet and the address of the server. Terminal opens a secure communication channel and sends the packet to a predefined server endpoint. Terminal may perform retry logic or buffering if transmission fails. Terminal outputs a transmission result status to its local log and, optionally, an acknowledgement indicator to the user interface.

### Step 9:

Server converts audio information into text information.

Server receives, as input, the response information, including audio data and metadata. Server forwards the audio data to an acoustic recognition processing unit. The acoustic recognition processing unit computes acoustic feature vectors (for example, mel-frequency coefficients) from the raw waveform and feeds these vectors into a trained speech recognition model that calculates probability distributions over phonetic or textual units. A decoding algorithm selects the most probable sequence, yielding text information representing the user's speech.

Server outputs the recognized text string and an associated confidence score.

### Step 10:

Server estimates the emotional state from audio and image information.

Server receives, as input, the audio data, optional image data, and session metadata. Server extracts audio feature information such as pitch contour, intensity, and speaking rate, and image feature information such as facial landmarks and expression codes. Server inputs these feature vectors into an emotion estimation processing unit, which may be a neural network classifier trained on labeled emotional data. The unit computes class probabilities for multiple emotional categories. Server selects the dominant emotional label, such as “anxious,” and computes an emotion score vector. Server outputs the emotional state label and the associated scores as structured emotion data.

### Step 11:

Server analyzes text information using natural language processing and the generative AI model.

Server receives, as input, the recognized text information and, optionally, a brief context summary derived from history. Server passes the text through a natural language processing pipeline that tokenizes, tags, and parses the sentence, identifying symptom-related terms and negations. Server then forms an analysis prompt sentence, such as: “You are a health monitoring assistant. Read the following statement and extract physical symptoms, emotional state, overall risk (low/medium/high), and whether to notify family (yes/no) with a short reason: ‘I have had a slight headache for three days and I feel anxious.’” Server sends this prompt sentence plus the user statement to the generative AI model. The model processes the input via transformer layers to generate a structured textual output listing symptoms, emotional descriptors, risk level, and notification recommendation. Server parses this output using pattern matching or delimiter detection to extract state information, risk information, and abnormality-related information. Server outputs these elements as structured analysis result information.

### Step 12:

Server stores state, risk, and emotional data as history information.

Server receives, as input, the analysis result information and the emotional state data. Server constructs a history record containing user identifier, timestamp, extracted symptoms, risk level, abnormality flag, and emotional state label. Server writes this record into a database table or document collection, indexing it by user and time. Server may also update aggregate counters and rolling statistics such as symptom frequency and average risk over a defined period. Server outputs an updated history dataset and acknowledges successful storage to internal monitoring modules.

### Step 13:

Server performs anomaly determination based on current and past records.

Server receives, as input, the new history record and the recent history dataset for the same user. Server evaluates anomaly rules or statistical models that compare current symptoms and emotional state to past patterns. For example, server computes the number of days in a recent window with reported headache and checks if this count exceeds a threshold, and checks if emotional state has shifted to a negative cluster. Server combines these conditions with the risk level from the analysis result information to determine whether an abnormal condition is present. Server outputs an anomaly determination result, including a boolean abnormality flag and an anomaly severity score.

### Step 14:

Server generates notification information for related persons.

Server receives, as input, the anomaly determination result, the latest history record, and contact configuration for related persons. Server decides whether notification is required and, if so, composes notification information. Server builds a concise description using the latest symptoms, risk level, emotional state, and relevant historical context, such as “Headache reported for three consecutive days with anxiety detected.” Server formats this description together with alarm information into a message body. Server outputs a structured notification object that includes message content, target addresses, and preferred communication channels.

### Step 15:

Server transmits notification information via communication interfaces.

Server receives, as input, the notification object. Server selects a communication interface such as a mobile network interface or data network interface according to the target address and severity. Server encodes the message into the format required by the chosen channel and sends the notification to the information processing terminal of a related person. Server monitors transmission status and logs success or failure. Server outputs a delivery status and, if successful, a record of the time and channel used for notification.

### Step 16:

User and related persons receive feedback and may initiate follow-up actions.

User receives, as input, any local feedback generated by the server and relayed through the terminal, such as a reassuring message or simple instructions. User listens to or reads this feedback. A related person receives, as input, the notification on their information processing terminal, such as a mobile device. The related person interprets the content and may initiate a call, visit, or further medical consultation. User and related person output behavioral responses that are outside the scope of internal system computation but are triggered efficiently and accurately due to the system's data processing and anomaly detection.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

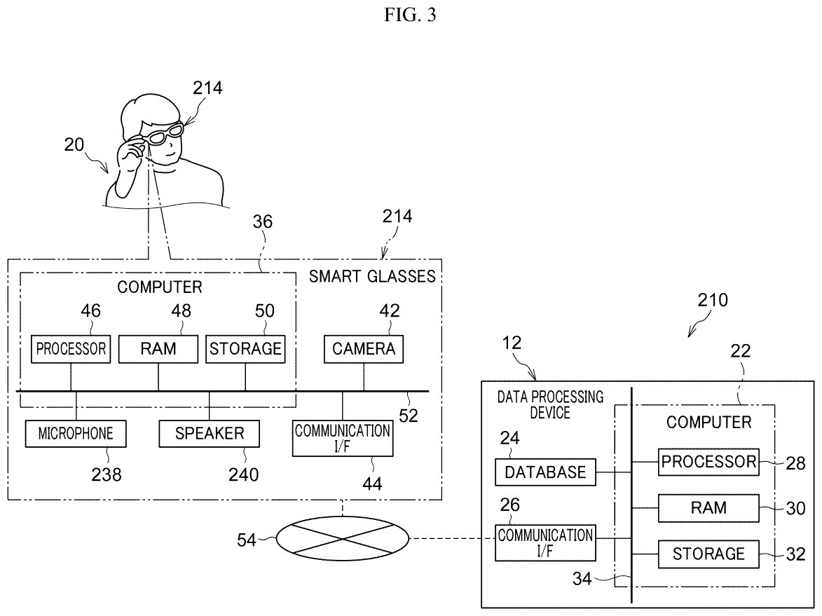

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

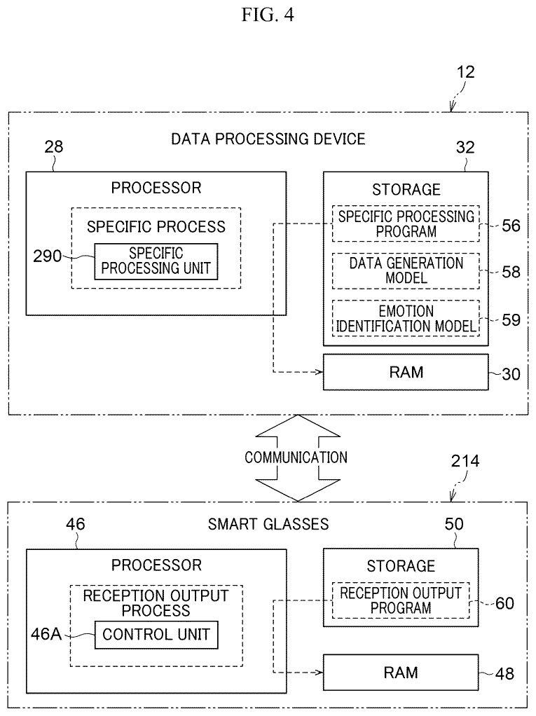

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

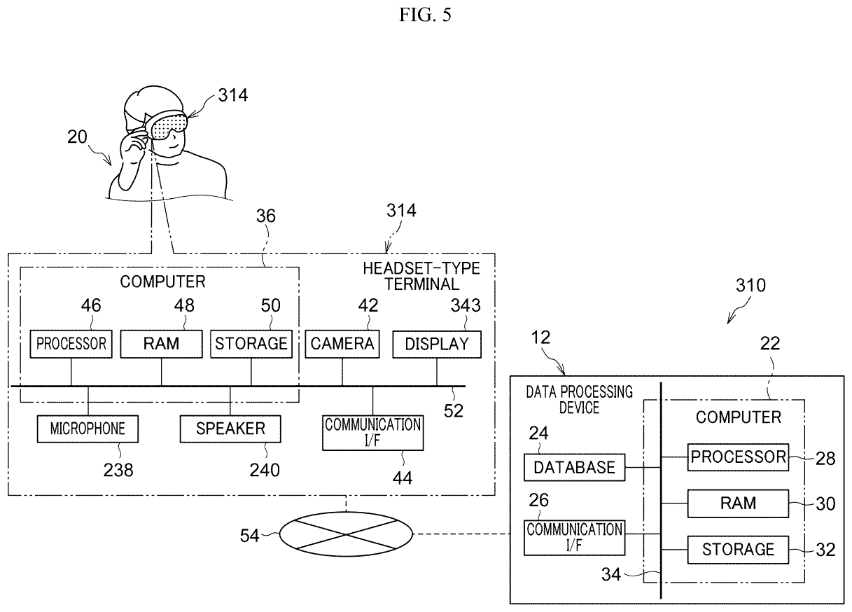

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

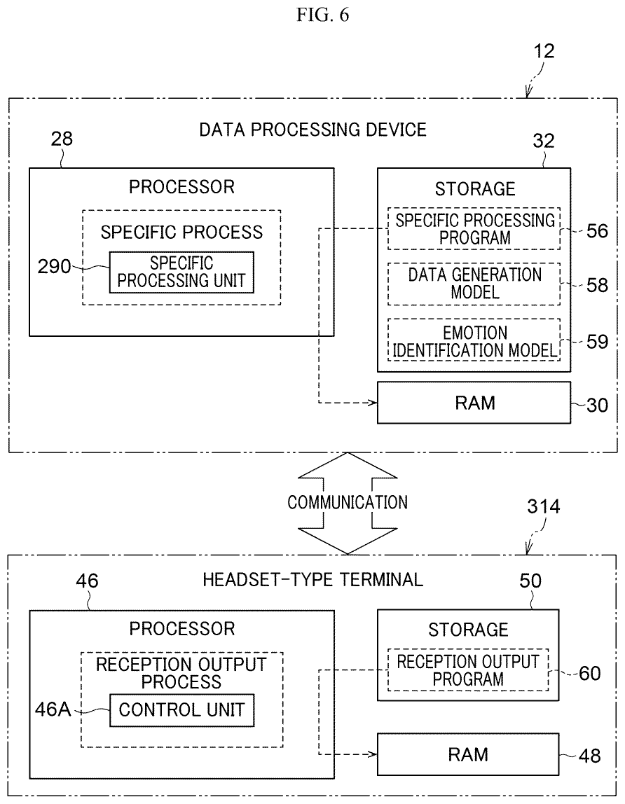

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

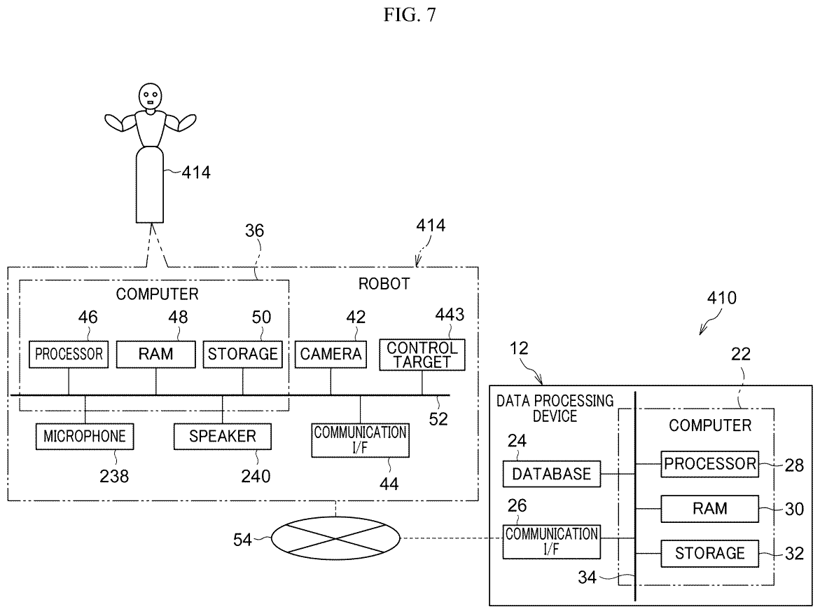

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

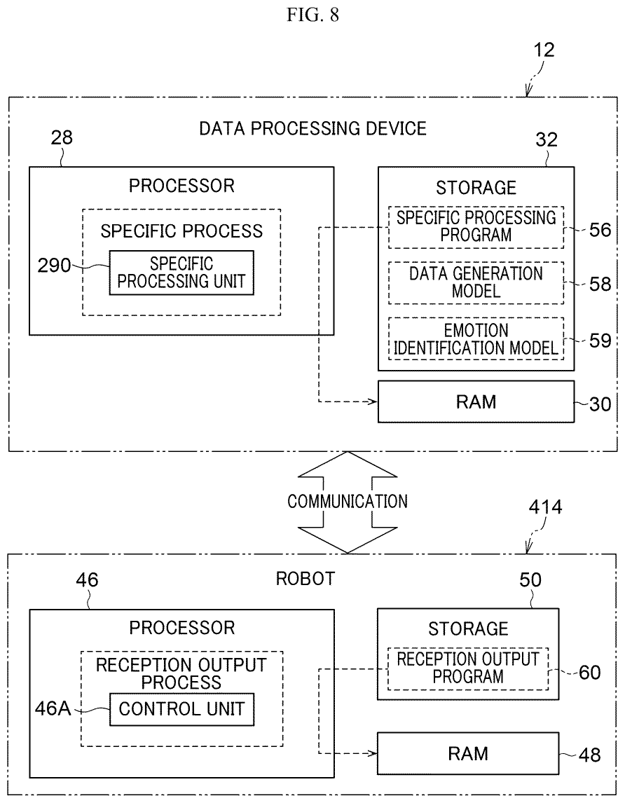

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

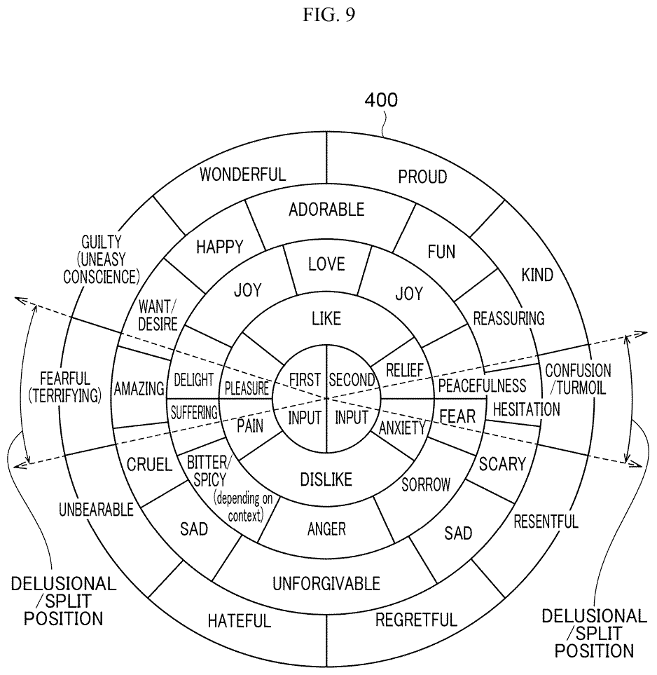

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

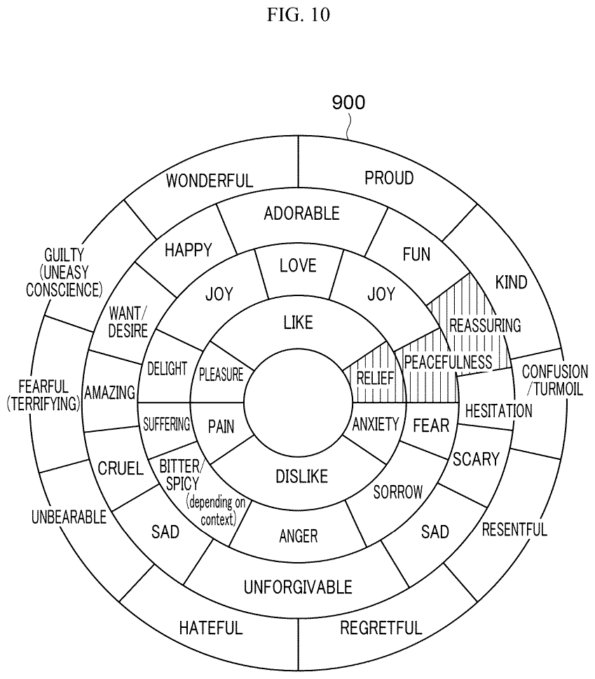

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### (Supplementary 1)

A system comprising a processor,
wherein the processor is configured to
receive, as input data for a generative AI model, history information including past health condition information and conversation history of an elderly user, and construct a prompt sentence that instructs the generative AI model to generate a natural language inquiry sentence including at least a prompt sentence and a question sentence for checking a current health condition of the elderly user based on the history information, and generate the input data for the generative AI model,
transmit the inquiry sentence output from the generative AI model to a terminal device via a communication interface so that the terminal device presents the inquiry sentence as audio or text to the elderly user, obtain response information in natural language from the elderly user via the terminal device, and collect the response information as text data,
analyze the collected response information by using a natural language processing unit, convert the response information into structured data including at least symptom information, severity information, and emotion information, and store the structured data in a database on a storage device in association with the inquiry sentence and time information by using a database management unit,
read a plurality of pieces of the structured data stored in the database, calculate at least symptom occurrence frequency, temporal changes, and deviation from a reference value by using data processing technology including a statistical processing unit and an anomaly detection unit, evaluate a change in the health condition of the elderly user, and generate an analysis result indicating presence or absence of an abnormal state and a degree of urgency, and
control automatic transmission of a notification message, via the communication interface, to a registered notification destination including at least a family member or a caregiver when it is determined, based on the analysis result, that the abnormal state satisfies a predetermined condition.

### (Supplementary 2)

The system according to supplementary 1,
wherein the processor is configured to generate a summary text by summarizing the history information before generating the inquiry sentence by using the generative AI model, and to construct the prompt sentence such that the summary text is included in the input data for the generative AI model.

### (Supplementary 3)

The system according to supplementary 1,
wherein the processor is configured to generate content of the notification message by inputting the structured data and summary information on the change in the health condition into the generative AI model, causing the generative AI model to generate a notification message including a natural language explanation sentence for the family member or the caregiver, and constructing the notification message on the basis of an output of the generative AI model.

### Application Example 1

### (Supplementary 1)

A system comprising a processor,
wherein the processor is configured to
generate, at a user terminal operated by an elderly user, generation instruction information for inputting a prompt sentence to a generative AI model in order to cause the generative AI model to generate at least one question sentence for checking a health condition of the elderly user, the prompt sentence including at least attribute information of the elderly user and a health-check purpose for a current day, and to transmit, via a communication network, a request including the generation instruction information to an information processing infrastructure on which the generative AI model operates, and to acquire, from the information processing infrastructure, the at least one question sentence generated based on the prompt sentence;
present, by using a display function or a voice output function of the user terminal, the acquired at least one question sentence to the elderly user, obtain response audio information from the elderly user as a response to the at least one question sentence, convert the response audio information into character information by speech recognition processing to thereby generate answer text data, and temporarily store the at least one question sentence and the answer text data in association with each other;
transmit, via the communication network, the temporarily stored answer text data to a server, and, at the server, apply a natural language processing technique to the answer text data in order to extract health state information including symptom information, emotional state information, and severity information, and store the health state information in a database configured in a storage device in a cloud environment in association with at least a user identifier and time information;
periodically execute an abnormality determination process in which the processor calculates a health risk index for the elderly user based on a plurality of pieces of the health state information stored in the database for different time points, and determines whether the health risk index satisfies a predetermined abnormality determination condition;
when it is determined in the abnormality determination process that the health risk index satisfies the abnormality determination condition, acquire contact information of a family member or a healthcare provider stored in a contact information storage unit, generate a notification message including latest health state information of the elderly user and abnormality content by message generation processing, and automatically transmit the generated notification message, via the communication network and by using at least one of a short-range text communication service, an electronic mail service, and a notification delivery service, to the contact information; and
generate, based on a result of the abnormality determination process, a guidance prompt sentence for input to the generative AI model in order to obtain a feedback sentence including a lifestyle precaution or a medical consultation recommendation to be presented to the elderly user at the user terminal, transmit the guidance prompt sentence to the generative AI model, acquire the feedback sentence generated by the generative AI model, and cause the user terminal to present the feedback sentence by display or voice output.

### (Supplementary 2)

The system according to supplementary 1,
wherein the processor is configured to include, in the prompt sentence to be input to the generative AI model, at least part of past health state information of the elderly user, symptom occurrence history stored in the database, and information relating to the health risk index, so that question sentences are generated in a manner personalized for each elderly user.

### (Supplementary 3)

The system according to supplementary 1,
wherein the processor is configured to generate, based on the health state information and the health risk index obtained by the natural language processing and the abnormality determination process, a control prompt sentence to be provided to the generative AI model, the control prompt sentence indicating a continuous monitoring policy for the elderly user and at least one of content and frequency of question sentences to be presented in subsequent sessions, and to dynamically change generation of the subsequent question sentences by the generative AI model based on the control prompt sentence.

### Example 2

### (Supplementary 1)

A system comprising a processor,
wherein the processor is configured to
generate, based on past health-related information of an elderly user and general health-related knowledge, a prompt sentence to instruct a generative artificial intelligence model to generate at least one question sentence for checking a health condition of the elderly user, and input the prompt sentence into the generative artificial intelligence model so as to obtain the at least one question sentence,
transmit the at least one question sentence to a terminal device for the elderly user via a communication network, and cause the terminal device to present the at least one question sentence to the elderly user by using at least one of a sound output function and a display output function,
acquire an answer from the elderly user through the terminal device as at least one of a voice response and a text response, and, when the answer is the voice response, convert the voice response into character information by speech recognition processing to normalize the answer as answer data,
analyze the answer data by using a natural language processing engine to extract health state indicators including at least one of a symptom, an emotion, and a time expression, and
generate structured data representing the health state indicators,
store the structured data and previously accumulated structured data in a memory device, and perform at least one of time-series analysis and statistical analysis on the structured data and the previously accumulated structured data to detect a change in the health condition of the elderly user,
determine that the change in the health condition is an abnormal state when a predetermined condition is satisfied, and, in response to the determination, transmit, by using a communication interface, notification information including details of the abnormal state to a related terminal device of a related person, and
update contents of a subsequent prompt sentence to be input into the generative artificial intelligence model, based on the detected change in the health condition and past analysis results, so as to dynamically adapt contents of the at least one question sentence to a current state of the elderly user.

### (Supplementary 2)

The system according to supplementary 1,
wherein the processor is configured to
store presentation time information and presentation history information of the at least one question sentence in the memory device, generate a presentation schedule for the at least one question sentence for the elderly user based on the presentation time information, and control the terminal device to present the at least one question sentence according to the presentation schedule.

### (Supplementary 3)

The system according to supplementary 1,
wherein the processor is configured to
execute a determination logic that evaluates a combination of a plurality of types of the health state indicators and change trends of the plurality of types of the health state indicators based on an analysis result of the natural language processing engine, calculate a risk level according to the determination logic, and control transmission of the notification information such that at least one of presence or absence of the notification and contents of the notification information is switched in accordance with the calculated risk level.

### Application Example 2

### (Supplementary 1)

A system comprising a processor, a memory, at least one terminal device having a communication function, and at least one communication interface,
wherein the processor is configured to cause a generative AI model to generate, based on a prompt sentence including an instruction content for creating inquiry information for confirming a physical state and a psychological state of an elderly person, at least one question sentence to be presented to the elderly person,
wherein the processor is configured to control the terminal device to present the question sentence to the elderly person as audio information and/or character information and to cause the terminal device to acquire, as response information from the elderly person, audio information and/or character information and to transmit the response information to the system,
wherein the processor is configured to convert, by using an acoustic recognition processing unit having an acoustic information processing function, at least the audio information included in the response information into text information,
wherein the processor is configured to analyze, by using an analysis processing unit having a natural language processing function and the generative AI model, the text information to extract state information relating to the physical state and the psychological state of the elderly person and to generate analysis result information including risk information indicating a degree of risk and abnormality information indicating presence or absence of an abnormal condition,
wherein the processor is configured to store, in the memory acting as a storage management unit, the state information, the risk information, and the abnormality information as history information, and to evaluate, based on comparison between current history information and past history information, a temporal change in the state of the elderly person,
wherein the processor is configured to estimate, by using an emotion estimation processing unit based on audio feature information extracted from the audio information and/or image feature information extracted from image information acquired from the terminal device, an emotional state of the elderly person and to record the emotional state in association with the state information in the memory, and
wherein the processor is configured to execute abnormality determination processing based on at least the analysis result information, the emotional state, and the history information and, when it is determined that the abnormal condition is present, to generate notification information including alarm information and to transmit the notification information automatically, via the communication interface acting as a communication processing unit, to an information processing terminal of a related person of the elderly person.

### (Supplementary 2)

The system according to supplementary 1,
wherein the processor is configured to generate, based on the history information stored in the memory and on the emotional state of the elderly person, the prompt sentence given to the generative AI model so as to change inquiry content for confirming the state of the elderly person, and to cause the generative AI model to generate the question sentence in such a manner that content of the question sentence is dynamically changed according to a change in the physical state and the psychological state of the elderly person.

### (Supplementary 3)

The system according to supplementary 1,
wherein the processor is configured to determine a content and at least one destination of the notification information according to the abnormality determination processing based on the analysis result information, the emotional state, and the history information, and to transmit, via the communication interface using at least one of short-range wireless communication, mobile communication network communication, and data network communication, the notification information to the related person of the elderly person.

What is claimed is:

1. A system comprising:
circuitry configured to:
generate, based on record data stored in a storage device, instruction data for a generative model, the instruction data directing the generative model to produce one or more data sequences adapted to a target entity based on context parameters derived from the record data;
packetize the one or more data sequences and transmit the packetized data sequences to a terminal device via a communication interface coupled to a packet-switched network for output via at least one of an audio codec or a display controller of the terminal device;
receive, via the communication interface, response data packets from the terminal device;
apply natural language processing to content of the response data packets to extract structured attribute data and store the structured attribute data in the storage device indexed by timestamp values;
execute, on a plurality of stored structured attribute data entries, a computation comprising at least one of frequency analysis, temporal trend detection, or deviation computation relative to a reference data set, and generate a classification output based on the computation; and
responsive to the classification output satisfying a threshold condition, construct and transmit a notification data packet to a registered network address via the communication interface.

2. The system according to claim 1, wherein the structured attribute data comprises at least symptom information, severity information, and affective state information extracted from the content of the response data packets.

3. The system according to claim 1, wherein the circuitry is configured to:
generate a summary text by summarizing the record data, and
include the summary text in the instruction data transmitted to the generative model so that the one or more data sequences are personalized to the target entity.

4. The system according to claim 1, wherein the circuitry is configured to:
calculate a risk index for the target entity based on the plurality of stored structured attribute data entries, and
determine whether the risk index satisfies the threshold condition by comparing the risk index against a configured threshold value.

5. The system according to claim 4, wherein the circuitry is configured to:
generate, based on the risk index, a guidance instruction for the generative model to produce feedback data comprising at least one of a precautionary recommendation or a consultation recommendation, and
packetize the feedback data and transmit the packetized feedback data to the terminal device for output via the audio codec or the display controller.

6. The system according to claim 1, wherein the circuitry is configured to:
generate content of the notification data packet by transmitting the structured attribute data and summary data to the generative model and obtaining a natural-language explanation sentence from the generative model.

7. The system according to claim 1, wherein the circuitry is configured to:
transmit the notification data packet using at least one of a text messaging protocol, an electronic mail protocol, or a push notification protocol via the communication interface.

8. The system according to claim 1, wherein the circuitry is configured to:
decode audio data received in the response data packets into text data using speech recognition processing, and
use the text data as input to the natural language processing.

9. The system according to claim 1, wherein the circuitry is configured to:
include, in the instruction data, at least a portion of past structured attribute data and occurrence history data stored in the storage device, so that subsequent data sequences are generated based on accumulated context.

10. The system according to claim 1, wherein the circuitry is configured to:
generate a control instruction indicating at least one of content adjustment or frequency adjustment for subsequent data sequences based on the classification output, and
dynamically modify generation of subsequent data sequences by the generative model based on the control instruction.

11. The system according to claim 1, wherein the circuitry is configured to:
periodically execute the computation and the generation of the classification output at a configurable interval based on a schedule stored in the storage device.

12. The system according to claim 1, wherein the circuitry is configured to:
store the one or more data sequences, the response data packets, and the classification output as session records in the storage device in association with an entity identifier and a session timestamp.

13. The system according to claim 1, wherein the generative model comprises a transformer-based language model, and the circuitry is configured to:
tokenize the instruction data into token sequences, and
transmit the token sequences to the transformer-based language model via the communication interface.

14. The system according to claim 1, wherein the circuitry is configured to:
estimate an affective state of the target entity based on the content of the response data packets, and
adjust at least one of a tone, a content focus, or a number of the one or more data sequences based on the estimated affective state.

15. The system according to claim 1, wherein the circuitry is configured to:
generate report data aggregating the structured attribute data, the classification outputs, and notification records over a specified period, and
transmit the report data to a management terminal device via the communication interface.

16. The system according to claim 1, wherein the terminal device comprises at least one of a mobile computing device, a wearable display device, a headset-type terminal, or a robotic apparatus, each coupled to the packet-switched network via the communication interface.

17. The system according to claim 16, wherein the terminal device comprises the wearable display device including a microphone and a speaker, and the circuitry is configured to:
receive audio data packets from the microphone of the wearable display device via the communication interface, and
transmit audio output data packets to the speaker of the wearable display device.

18. The system according to claim 1, wherein:
the circuitry comprises a processor, a memory storing a program, a communication interface, and a storage device,
the processor executes the program to implement the generation of the instruction data and the natural language processing of the response data packets,
the storage device stores the record data, the structured attribute data, and the classification outputs, and
the communication interface exchanges data packets with the terminal device and the registered network address over the packet-switched network.

19. The system according to claim 18, wherein the storage device comprises a database that associates entity identifiers with structured attribute data entries, session records, and notification logs.

20. A method performed by circuitry, the method comprising:
generating, based on record data stored in a storage device, instruction data for a generative model, the instruction data directing the generative model to produce one or more data sequences adapted to a target entity based on context parameters derived from the record data;
packetizing the one or more data sequences and transmitting the packetized data sequences to a terminal device via a communication interface coupled to a packet-switched network for output via at least one of an audio codec or a display controller of the terminal device;
receiving, via the communication interface, response data packets from the terminal device;
applying natural language processing to content of the response data packets to extract structured attribute data and storing the structured attribute data in the storage device indexed by timestamp values;
executing, on a plurality of stored structured attribute data entries, a computation comprising at least one of frequency analysis, temporal trend detection, or deviation computation relative to a reference data set, and generating a classification output based on the computation; and
responsive to the classification output satisfying a threshold condition, constructing and transmitting a notification data packet to a registered network address via the communication interface.
